SEQUENCE LISTING (28 sequences)

SEQ ID NO: 1 - DNA, 24949 bp, Homo sapiens
  misc_feature (1)..(24949): Genomic Stat5A DNA sequence (from Genbank Accession Nos. AJ412877.1..gi17975717 and NC_000017.9..gi 51511734)
agtgggaaaggagaggagagggactatacttcctcctccctggggccccctgcagagcatctgggaagcaaggcttccctacatcctccatgcacccccttagagttttcaattcctttcctcgtgatcctgccaactaagacactgtgaccacacagagaaggtggggagaacgcagacattttggcttctgcagctttgaagttctttttttttttttcctctgaagttaaaagaatgaaactgggagaggtagtaaggggcaagaaaggagagtggaaatggagagaaaagggcagctctgagaagcagctggggagggaggcagatgagaatgcaccccccccaacagaacatgcagtcttggcccagctgtgctgtgagtgggcagctgggctggcccctcctctggtgctgccaacccgctgccaggcagaggggaggcccagaggagagggaagctgggcaaaggggatggaaggcgtccagcccgaccttaccaaaccccttgggcctcgtgggaaggggcctcttggagagggggactgaggctctagacaggatattcactgctgtggcaaggcctgtagagagtttcgaagttaggaggactcaagacggtccctccctggacttttctgaaggtagaaccagcctcataagtaactaggctgggtgaacgggggcgctggctagtttatggatcacagtcggctggtgaggccacgtgcctactgtgtggccctgggtggccccgggcaagcccctttccctctcaggacttccatttcttacctgcaaaattgtggagagggggagggctgaaacacatgactgccaagattctttccagttcctccgtcagggttgagtttagatggccggagtaaaagaaggagggaggtgctgcggtggtgggggtgatcttggcttcactagaatccccagttcttcccctctctacagttttgtctctgaggtcacaaaacctgtggcccccaagacacacatgcgcacacacgcgcgtgcacacacacaccccacacatttattttttaatctaggggctcaaaagatgacacgcgccagagctggaaggcgtcgccaattggtccacttttccctcctccctttttgcggatgagaaaactgaggcccaggtttgggatttccagagcccgggatttcccggcaacgcccgacaaccacattcccccggctattctgacccgccccggttccgggacgctccctgggagccgccgccgagggcctgctgggactcccgggggaccccgccgtcggggcagcccccacgcccggcgccgcccgccgggaacggccgccgctgttgcgcacttgcaggggagccggcgactgagggcgaggcagggagggagcaagcggggctgggagggctgctggcgcgggctcgcgcgctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgggtgacccggtggcgcgtcctcggcggcgcgccgagaggggacactctactgccgccccggcacctccggcacccggagctcgacggccgggcgcagcgcggggatcagtccccggcactgcgagggagttggcccagcgcagacgagggacggggggacgtggggacaggggtcggggatgaaaggcagaggccagggagggcgccgtcctggcacgcctcggagagggagcacctgtcgggctggacccggggaggcactagtgcacagatgggggagcagccgaagaggggagcgcccaagtcctcagccctgtctcctgaacagccccagccacgtgcccgaaaagccctagccgtcgagtgggagccgtcgtggcgctggcctaacttcgggttggaatatctccccttcccagaaaccgggcccgaggtgggctgggggatccttgcagccctctccagggggcctgtgggcctcccgccgtcaagtttctctttcagtttctggccgtccccctcactagggaagctccggtggggcgaggggagaggaagacggggagggcacctgcctctgcagagcaccttcccctccccttcagcgggttccttgttggaggggatgggcggccctccactcctcaccatctctgttcccgcacaggaaagctatctgtgggaggggaggggcctggcctggccgcggtccagggataggtaggcatggcaaggccaggggcaggcccggttccttggcctctgcagaggagagcgcttcagcgctcggctcgccctaggtgaacggccatggcgggctggatccaggcccagcagctgcagggagacgcgctgcgccagatgcaggtgctgtacggccagcacttccccatcgaggtccggcactacttggcccagtggattgagagccagccatggtaggcacgtcccgccctgcccctcctcagagggtccctaccatccaggccctttggcctctagttttactcatggtggtttggtgtgactctggtcctgcctgtccttctttgtgcggaaggggtggtgcccctgggaaaggggaagcctgggaggacagagaatgtttttagactcggatgtctgtggagctgctgggaacaagtcttgtgggccctggagtgccctcggtcatgagcctggggtttccactttattccagctccctgacctccttgcccaaggaggtgccacagtaggtttttctcttctgccctgccccaagggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccaggtgggtgtaggctctgggccacctacggggaggaagcatcatcttgttccagcatcagaacccctgggttttttcctggcctggccactgactcaccatgtgaccacgaacttattgggaacttttcttcgatttcccccaccccctctttctaaattcgacctgtcggatttctttagcatattgcaaagcaagggtgggaggtgtttgttgccaacagagagagatgggtgaactggccttatggaagcagcagaagggatggagggagactgagtcaggattcctatgttggagaagggaagaccagcagactctagaaatgaggccaccagacctccctcttggggtcttttaaaggtgagagaggagccatcccagagacaagagctggacacaagggctttgaattgagggtttagaaataagatgtagcttctttacttgaggggagtgggtatggaggaagagagggagtttttgtggaagcaggaaaggggtgtgggtagtgatgacagtcaagcacttattggaactcacgtatgtttcctagagagcccttcaatacagacagcagggtgccagggaaatacaagtccatcccagaagagggggaaaacaggagaagcctgaggggaagcaatataggaggccaggggcaggatggtgggaagtaccttgaccttggcatcagatgccctgagtctggagtcatctgctctctcacttggttgcgtctggacgagtacataatgtctgagcatcagttttctcatctgtgaaatggggatcatagtttctgcttatgtcctagagtttccatgctgttagtgccttgatcccccaccactttttggcaccagggaccggtttcatggaagacaatttttccatggaccgggattgggcagggaggtggtttctggatgattcaagagcatcacgtttattgtgcactttatttctattgttattacattgtgctatgcaatgaaataattatagaactcaccacaatgtaggatcagtgggagccctgagcttgttttcctgcaactagacagtcccatctgggggtgatgggagacagtgacagatcaccaggcattagattctcttaagaagtgcataacctagatccctcgcatgtgcagttcacaatagggttagcattcctatgggaatccagtgccattgctgatctgacaggaagcagagttcaggctgtaatgcgagcaataggcagcagctgtaaatacagatgaagcgtcactcacttgctcaccgccgctcacctcctgctgtgtggtccagttcctaacagaccaccgacctgtaccagtccatggcccaggggtttgggacccctgtgttacagtcaaaagatacggtagattggaagtgcttcataagactgggtatgatggctcacacctgtaatcacagcactttgggaggccgaggcgggtggatcacctgaggtcgggagttcgagaccagcctgaccaacgtggagaaaccccgcctctactaaaaatacaaaattagccgagcgtgatggcacatgcctgtaatcccaactactcaggaggctgaggaaggagaatcgcttgaacccgggaggtggaggttgcagtgagctgagatcacaccattgcacctttagcctaggcaacaagagcaaaactccatctcaaaaagaaaaaaaaaaaaaagagaaagtgcctcataaactgtgggtttttaattcgctccaaaccagatatgaagatcttggaggggaagatgaggatgaggaacagagatatagaggtgtctggggatagttcctgaggctccctgggaaaagctgaggcagaggctgaaggagaggaagggctgggcatagcatggggctggcatggctggagcagaggatggcgctggaggctactgttggattctttcagaaaacatatgaccgctgccccctggagctggtccgctgcatccggcacattctgtacaatgaacagaggctggtccgagaagccaacaatgtgagtgtcccttggggatggggaggagtgttgagaagtccctccatatgcctttctctccagaaacaactgtgtttatataaaacagcagcacgaggagagcaccaagaagatgagggacgtgggcaagtgtaagaggtggcagcattgcacgccggggtggagtgaccccctggatccagtccctggctctaccacttactagctgtgtgacttgagcaagttgcttaatctctctgagcctctgttcctgtttttttttcttttttcttttttcttttttttgagacggattcttgctctgtcacccaggctgcagtgcagtggcgtgatctcggctcactgcaagctctgccttccaggttcacaccattctcctgcctcagcctcccgagtagctggggctacaggcgcccaccaccatgcccggttaattttttgtatttttagcagagatggggtttcactgtgttagccaggatggtctcaacctgacctcgtgatccgcccacctcggcctcccaaagtactggggttacaggcgtgagccaccacgcttggcgcctactgttttttcccccccgagatgcagtcttgctctgttgcccaggctggagtgcagtgatgtgatctctgctcactgcaacctctgcctcccaggttcaagcaattctcttgcctcagcctcccgagtagctgggattacaggtgcccaccaccatgcccggctaattttttttgtatttttagtagagacgtggtttcactatgttggccaggctggtcttgaactcctgacctcatgatccacccgcctcggcctcccaaagtgttgggattacaggcgtgagccaccgtgcccggcctgttcctactcttgtaaaatggagatagcaatcatacctatctgtgtcagaagaactaacacaggcaggctcagagaagtgaggcccagggaggggtaacagggacacatctccccagttctgaaataataaaggcactgggcatctacatggacaatggcacagtgctgtactatctgcaatagagcaaaatgaatttcacaacatgacgtgacagaaatcttagaacagtgaccggagcacaggtcatatttattatttctttctttatttgtttgtttgtttatttatttttgagactgagtctcactctatcatctaggctggagtgcagtggcgtgatctcggctcactgcaacctccaccccttgggttcaagagattctcatgcctcagcctcccaagtagctaggattacaggggcccaccaccatgcccggctaatttttgtatttttagtagagacagggtttcaccctgttggccaggctggtctcgaattcctgacttcaagtgatccacctgcctcggcctcccgaagtgctgggattacaggcatgagccaccacgcccggcctattacgtatttattgttgtggacatggatttcaggatttactcagtagacatttattgaggtcttactgtgtgtccgatgctgggctatcatcagagaaacacataaaggcctggttcttgcctggaaggaattctggcagtgatggattctctagagatgagctgagtgctaagggggcctaggtggaaaaagcacccaactcagattggggcaggctggcaggggagcgtcctgtggacaggcatgtctgagctacagtggagaaaggcaggagaggagggatgaattttctaatgaaaagacagcagctgcgagagcaggagggcatgaatcttcttatcacattggaagacttgtaagtggtcagtgtaactggggagaaggaaccaagtggggagttgccatggataaaaatagatagagagggccaggcgtggtggctcacatctgtaatcccagcactttgggaggccaaggtgggcggatcacctgacgtcagaagttcgagaccagcctggccaacatggtgaaacccagtctctactaaaaaatacaaaaattagctaggcgtggtggcgggcgcctgtaatcctagctactcgggcggctgaggcaggagaattgcttgaacccaggagggggatgttgcagtgagctgagattgcaccacttcactccagcctgggcaacagagcgagactctgtataaaaaaaaaaaaaataggggttagaaaccaggtcacaaagggtcttccagcacagcccctgtgtccctgacttgttgccatcacagagctatctgcaaacctaaaaatgacttcagcctcattgacaaatccatgtccatggtgctggtgctgtcatggtctgggagctcattccttcctccaaccatacaactgttcctgaacacccctggcattctagggcttggggattctgtagttcacaggaaaggcaaggtcctagcccttgggaacctcacatccttgtgcagggagacagacagaaacatgtaagcaaatacaagaacaacacacttttagataggactactgtgaattggctcgtcagagatgacccctctgcaatggtaccagttaaagtgctttgcctcaggaggatctgatgccagaatgttccagactgttggaacaataagtgcaagggccccagggcaggaacatgcctgtgtgttcaaggaacagaaacagaaagcacacattcccagcgagctggggttggaggtaggcagggctatggccagcagacaccatgggaaggggtttggattttattctgagaacagttcattggaaagcctctgaggacttgttcctcttcatccatgtgctccctccctttgtgacctggtttctacccctctctctctattttttttttttttgaaacggagtctcgctgtgttgccaaggctagagtgcagtggcgcaatctcggctcactgcaacatccccctcctgggttcaagcgattctcctgaatgtgctgaaggaatggctggatccaaggggagcttgttgatgcctcccctctaacttcatttcggggtctccaggatgcctgtgagagtttcggcctcttccctcttccttcagtcacaagggggtcttaagtgagatggtgtgtgttcacacagccctgttcctccctatgtccctaggatctactgcagtccatggtattgagtcagcaaataaatgtagagcgaggggtcatcatcattagtgggaaaacccaggtgacacctgggacgtgttgagttctagtccctgggaggaaattagatggaccctgtttggaatactctagtggagggcagctctgacatagatatttggactttgaaggtgttactatctgccgttatctgccaggtcacctgtttctcctttctctttcccttttctatcccaattctggcaaatgactccttctgatgcaaatgatccttccttcttgccctagtttcccttcttaccctagtttggggtttggggtttggggtctgtagtattggtgtttcctaatgcctgtggtcttctcccatcctctcttccccgagttatttccattccatctgtctccagtgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaaggtgaggcgcggtggaggcagtgtgcatccggagggtgggagtgaggaacattctatggactcctagagggtagagctgggtcagtgtccatctcctcagcactttgcccaaagcctggtagaagcaatggctcccggtgattgagcccctgccgtgggtcacacttggcacacattatttaattcaaccctctcaacaagcctatgaagtgaacagttgtgttatccccccttttgcagatggtaaaagtgaggttcaaagagaacaagtaacttggcctaggttgcctgtaaattacagaactggatctcaactagagatatatcataaaactcctgtactttattaaccagtattctctccttaatggtatgtattctgtaaatatcgacacaaataggtaaatatgaacagtgctccctaaccagagggaggacttggattggaagtcttctggttgaaccaggctgggagaggtggaacttccaaagtgggccttcttgagcctcacggataggatgtgggcctgggggaaggggtgacagtagaaaattctcctgtatcctaggtctgtagttcgccaatggctcccatgataaagatgtggggaaattaaaaaatcaaattagcttgaaaggaactatccccaggtacaatttgaggagtcatgggacatggcccatctattaatatgtcccattccagactgaccccagaaatgtaacacctcatcttagtgagaacaaggggtgtcaaactgctttttcctttactctttttaatctgatttcttaaggtagaccttaggtcattgattttagacctttcttcttttcttttctttctttttcttttcttttttttgagacaatcttgctctgtcgcccaggctggagcacagtggcatgatttcggctcactgcagcctctgcttcccagcttcaagaaattcttgtgcctcagtctcccgcatagctgggactacaggtgcaggccaccacgcccagctgatttttttagaattctattgtagctatttaagctctcttcctaaccctgcactgtcagtgctttgtgtacactgagaccagcacaatgcctgaatattgcagctattatgtaaatacttgcaggataattctaaatgtttgtttgtttgtttgtttatttattattgcccaggctggagtgcagtggcacgatctcatctcactgcgacctctgcctctcaggttcaaactattctcatgcctcagcctcctcagtagctgggactacagacatgcaccaccatgcctggctaattttttttttttgtagttttagtagatacagggttgtgccgtgttgtccaggctggttgcaacctctagcctcgagtgatctgcctgcctcggcctcccaaagtgctaggattataggcgtgagccacggtgcccagccaatactgacaggataattctaagagaatttcccctcatctgaatctatcccagactggccctagaagaagggcagctccttctgagaatggaaacggaagattttgggcggttattcctacccaagtccttagactgtcgacagtatttctgatcccgtgcctggcttttctgtttcctgtgcatggtggcggattaggtttggattcaggttgggtttggatctatcatgcagtgtcttctcagtcccatttgtcaagttcactcagcagaaacacagactattgatctgcttttgccagggaaggctcagctgcctccagtggagtagggaaattgcttcccgtctccctggcctccttcctaaaagcttctgcaaatttccaggagagccctaagtgggtcccttgcataggaaggctgggcccaggtgttgggatcaccaccaccctccctgtggctgggcaggatacccgaggtagccgtgcagacacctcggatctgtccgtgcctggatgatcctcttcctgctcgatacctgcagtgagattggagccatatttctgagttctagctctgggctgttcctggctacatagagttgcttttgtctgtcccagcttgtgcatcccatgggtcagggctggggtcagagtgacgtggccctcaggggccagccaggagctcgaccaagacatgctggtgcgcgcatcttctgactcagctcaagcaaggacgctgggctgttgagaaggtgatgggaagggattaatagtcccagtgtggtggagacgggctgtcgctgggagggtggggtgggcctgcggtggtggttgtaactgtggacatgtttactttcttagcctgggttattgatacacaggcgcccttgtgttccacatcccctgtgtcttacgtatttcataatggatacttttttttttttgagatggagtcttgctctgttgccttggctggagtgcagtggtgctctcggctcactgcaacctttaaccccgggttcaagcaattctcctgcctcagcctcctaagtagctgggattacaggcatgcgccaccatgcccagctaatttttgtatttttagtagagacagggtcttgccatgttggccaggctggtctcgaactcttgacctcaagtgatctgcctgtctcagcctcccaaagtgctgggattacaggcgtaagccaccgagcctggcacatgatggatacttttttttttttttttttagacagagtctagctctgtcgcccaggctggagtgcagtggcccaatctcggctcactgcaagctccaccttctgggttcacgccattctcctgcctcagcctccctagtagctggaactacaggcacccgccaccatgcccggctaattttttgtgtttttagtagagatggggtttcactgtgttagccaggatggtctctagccaggatggtctcgatcacctgacctcgtgatctgcccgcctcagtctcccaaagtgctgggattacaggcgtgagccactgcgcccagccccatgatggatactttttaaaagctagtgaggcctgacttattccctgtcccgccatcaatgagactcctcacagatggtgtggagggaagccgcctcccttcggcctccctttcctgagagaatcttggtggctctgcatccccagtggccctggacacgctctccctctgcggtctgtctatctgtctgtctcccctggttggtcggctgggctcacagtgagcccacctggacaaacatgctgggccccttccagcccctcaagaatctcatcctcccctgaggagggactgctgtgctcagggacgagacagagccgccggggctggggttccctgaagaggccttgctgcgggagggatggggccgagcagctgcttgtctctgtcacctcttctctctgtcctgttccccgttaaccaagaagcacgaacctcacacagagctcacccatgtcaggcagctttctggcatcttccatgtcctcatgtatttaatcttcaagcaccccactgaggcaggtgctgagaatctcccgttttacacgtggggaagctgaggcacaggaaaggaaagcgatttgcccaggttcacctgcagtcaagggcatgggcacacttacaccccagacagcctggacccatgctgggacgcgagaagagggctggcagccctgtgaggcaggcgggcaggaggcaggcaggcctcctccccagctgccccagcacccgtgtgcccccagaggggacggctcagagtcaggaggctggtgagcagggcccagaggcgcctcctgaggcgccattcatcttggcccccctggctgtcaggaaccccgacttgctcttgatgcagtgtctgagcctgggagggtctcgctccagaacaggtgagtgggcttctctaggcctcctgttggacactaggaggtgatggtcatggtttcctcttctctcctctagctcagtttgcccagctggcccagctgagcccccaggagcgtctgagccgggagacggccctccagcagaagcaggtgtctctggaggcctggttgcagcgtgaggcacagacactgcagcagtaccgcgtggtgagtggggtcctgggcctctcctgggcgtgggtgccatgaagtcagtctctggggacccgagggagggctgggaccagcatgagagcagaacctgggagggcaggaggccttttttcctgggggccttgcgaggcagagcagcttggggagagggaacggcctgggccctgccctgccatttcccagcgaggccctgcggtgttctctgggagcccagaaggggctgctctcctcccttccctcaggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtaatggtgtggggcggccagcgggcagggcaggaggggctggtggggaccccggctgcttctgcgtcctgtgctggctcctgcagcaggccccctccatcctgggacagctcttgtttctccagggagcctggttcctcaatcagactttggtccccatcctgtgcacctcccccaggaagggggctgctgtcctgggggtgggatggggctcgggtgtgtggggtgatgcttgggctgtttgggcctagtcagggtcgcccctcctgtgtacgtctctaattctgggaggcagggagctctgctcttcccatgggtgggaagtgtggcgaaagcacagagccttcctgggggaacgggagctgtgtcttggggcctggcgtctgtgaggagaagccattgtcctcctgttggccttggggctctcgtgcaggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccaggtgactgctgcctgtttgccatgcccaggagcttggggcagctcctgcctgcgtggggggagctgcaggtgccttccagaccagcagatccacttcctgcctctcatccctcccaactccatctccagttgctgtggctgctaggtgccgcccttgcccagtttctcctgtggacactgcaagagataccctgggccgcagcccatgccagctgctgtgttatctcactcccctctccatcctctccctggactctcacagcttccccgggaacagaggttgtgcccgagctcatcacctcctgagccccatgggaggcagccccacccccaccacagagggactgagagccctttcccctgcgccaacccctcatcgtgtgtctgtccctgtgtcccatgcagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaagtaattgtgcctctcccttcccctgcccaagcttaggtgtgggggacctgcaccccccgctttgtccttgcatccagctatgtcttgtcccctagttcaccgtgttgcccaggctggtctcgaactcctggactcatgcagtcttcccacctcagcctcccaaagtgctgagttgataggcgtgagccaccacacccagccgagtatccgttctttaccctggggatggtgtggctgccggagagctgaggctcactgccactgcccagcattgtgagagactatcacaccactttctcctgaaagtgtattgcttcacaccattgtaaattcaaacaatcatgtcaaaccatcattaaatctgggacagatggtacactgccttcctcctactcagatgccttccctgtccctgcaacaagcttggtgctaggctagatgccgtggcttacccctgtaatcccagcactttgggaggccacggtcggaggatcacttgagcccaaaagtttgagttggacaatatagggagaccctcatcgctacaaaaagttttaaaattagctgggcatggtggtgcacgcacttgtagtcccagctactcgggaagctgaggtggtaggatcatttgagcctaggaatttgatactacagtgaggtatgattgtgccatggcactctaccctgggcaacagggtgagaccctgtctccaaaaaaatcacacaaagtatggtgttgtagctgtgttctagaacctgtgctacgcactgaggacaaaactgtgatcccaacagaactgggctgtcttctcgcccagccccttgagcagcagagcaggcagcagatgctgggaaatgcctgatggtgtagccatgtggcagggggatgtgtagggcaccgagaaagggggtcttcttgcaggattaaagaaatgacagtcttaggctcctggtggaattggaggtagctagtgatagggagggaagtagcagagagggaggactttcaggcagagcccatgtgccacatcctgagggaagagtacagagacattatgtgtagcagagagtggagaagtagtgtccagtgtcagggcaggccacccacagcgtatgcaggggatgaataaatgagtgaatgagtgatgtggaagacatggccccatcccttgtcatccaattaaaaatgtttactgaggctgggcgaggtggctcacgcctgtaatcccagcactttgggaggtagaggcaggcagatcacttgaggccaggagttcgagaccagcctggccaccatggtgaaaccctgtctctactaaaaatacaaaaattagccaggcttggtaacgtacacctgtagctccagctacttggacctctagtcctaactaccaactactctgaggcaagaaaatcacttgaacccggaggtggacgtggcagtgagccaagattgtgccactgcactccagcctatgacagagcaagactctgtttaaaaaaaaaaaaaaggccaggcacagtggctcacgcctgtaattccagcactttgggaggcccaagcgggcggatcatgaggtcaggagttcgagaccagcctggccaacatggtgaaaccccgtctctactaaaaatacaaaaattagctgggcatggtggtggaagcctgtaatcccagctactcaggaggctgaggcaggagaattcctcaaacccaggaggtggaggttgcagtgagccgagactgtgccattgcactccagcctgggcaacaaaagcaagactctgtttcaaaaaaaaaagtttactgatttcctaccatgtgatctggggtgaagatgaagacaaatgaccttctcactgcctttgagcttgcattgcagctggggagactgatgataatacactttaaaagatcaggcagggccaaccagtgatagacactggaaaagaagttaagacagagcaatctaggctgggcacagtggctcacgcctgtaatcccagcactttgagaggctgaggtggaaggatcgtttgagatcaggagttcgaaaccagcctgagtaacatggcaaaatccaatctctactaaaaatacaaaaattagccaggtatggttgtgtgcgcctgtagtcccagctacttgggaggctaaggcagtggaatcacttgaacctgggaggtggaggttgtggtgagctgagattgcaccatcgcactccagcctgggcaaaaagagcaaagctctgtctcaaaaaaaaaaagaaaaaagagcaatctgctagcgttggagatgtgtttaaggcgggggtgaggaaaggtcagcttggtaagcttctccagataagtggtccaggaaggcctttctggggagctatctgggctgagacctgaaggaggagaaggctaagagtgtttggggtgacaggaagagcaagggccaatgtccaagcctggcatgttcagcagacagccagggccaacggggcttgcatgtcctgagtgaggggagaggaattcagtgaggccaggggggtgggtaggtgttgaccatagggatcctcagagactgtggttgtgatttttatcttaagtgcattagaagctattggaagtccccaaagccctcacatcaatcttggtatcgaggaatttctaatgatagatccagacctcaccactggagaccttgccttgggtgctgggcaccaggttgatggagaagtcagtaacccagaaagacgccaagaaacactcttaggggatacggggcaggggctgctggcagggctgacctgagcgaagaccccagcccgaggtgtggacaggaccatgctcctggcctggggcccatgtggagctgggacccccctctcctttgcagcgagtgcagtggtgagatcctgaacaactgctgcgtgatggagtaccaccaagccacgggcaccctcagtgcccacttcaggaacatggtgaggacggggcccaccctcggagggcaggtctgcccagagctgagtccttgtaagcagccgccatctccctgttcccctgtcacctcccaccctgcagtcactgaagaggatcaagcgtgctgaccggcggggtgcagagtccgtgacagaggagaagttcacagtcctgtttgagtctcagttcagtgttggcagcaatgagcttgtgttccaggtgaaggtgagacccccagccctcctgcccccactgctccaggtcacccaagaggtggaggggcctgcctcaggactcctggcagcaatgccatcggacagcctgctttgactctgtgggtccctgtttgataggttataatctgggactaacccagacaatttaggcaatgggctaagaagaagtcggttgtagtttttgtttgtttgcttgtgcctacattttgctgggaggggcttgtgtttggaactgtgggtgaggaaagtgggttgggaccaggtgtggtggctctcacctgtaattccagcattttgggaggcagaagagggaggactgcttgagtccaggagttcaagactagcctgggcagcatagtgagaacccgtatctacaaaaaaaagaaaaaagttgggcatggtggcctgcacctgtagtcccagctattctggaggctgaggcaggaggatcacttgagcccaggaggtcaaggctgcagtgagctgtgattgcaccactgccctccagtctgggcgaccgagcgagaccctgtctcgaagaaaataaagcagattgcggccatgggtggtgggtcttgcctgtaatcccagcactttgggaggccaaggagggtggatcacctgaggtcaggggttcgagatcaacctgaccaacatggagaaaccccgtctctactaaaaatacaaaattagccaggtgtggtgacacatgcctgtaatcccagctacttgggaagctgaggcaggagaatcgcttgaacccgggaggcagaggttgtggtgagccgagattgcaccattgcactccagcctgggcaacaagagtgaaactccatatcaaaaaaaaaaatacataaaaaaaaataaagcagattgggcatgttgcctaagcagagggcacgtggtggtcacgccccatcaacttgggttcctttgactcctgtagactctgtccctacctgtggttgtcatcgtccacggcagccaggaccacaatgccacggctactgtgctgtgggacaatgcctttgctgagccggtgagtccccgtgggagccctaccccagcacccccaggccctaggactcacctggggtcagccccaccccttgggcccctgctgagtggtccttcctcaccccagggccagcccagaggtgaggtgaggccagaagggctggggtgctgggcgcctgccttccaccatggccttggcactctgggttccaggttcctggactggggtctgtgaggactgtgagcttggtctttggatcctggtctgggatcctcctttggcagatggcttgggtccttgctctctgttttctgtttcctggtctgcatctctgtccctgcatgcccccaccccctgcatcggttttcttccctgcaggctgggaaaaaaagtgcaagctagtatataaaagccgccgcatttcctgcccttgcatttgtgtcacctttctggatctgtctcagtctcctctgtctctaggtgtctgtgtatcttgtgtgtgtgtgaatatttccaactccctcaatctaccttttcccctctcttgtctccctctcagggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggtgaggggcccagctgccagccgcccaccagggcccaccggggtctgttgctcctccacctcacagacaggttactgcctggggctagcaccccactctccacccccaaccactctctcctacaaccaggagagatgggggcccctagcctcaagaaatgcaggcaggggctataggagttgtgcttagttgtgaagccaagagcgagatgacggagggcccagggctgggagtcaggatccttgggttctgggcctagatgtgtttgctaattttcttttttttttttagagacagaatctcattctgtcacccaggctggagtgcagtggcgtgatttcagcttactgtaacctctgcctcccgggttcaagtgattctcgtgcctcagcctcctgagtagctggaattataggtgcctgccaccatgctgggctaatttttgtatttttagtagagatggggtttcaccatgttggtcaggctggtctcgaactcctgacctcaggtgatccgcccacctcggcctcccaaagtgctgggattacaggcgtgagccaccacgcctgtggctctggctctccttctgcctctctccttgctaatttttgaagacccatccattctctgtgcctcagtttccccacctgtaaaatgaggggatgactttggaggaatgactttggaggccccatccagcctagattattctttacacctcttactcccacccttgggctggtcttggacccctgtgtcttagaagtgagtttcctctctgttttcctagggtacaagttgggggctgtgtcactttatggtctgctggagtgtggtggcaagcaggggtgaagaactgggagaagactgggtgggggcaggagggccctgactttcctgggccacctgtgacctggggcaccagccctgactcgggggttcctgggccctcaggagaacttgccgggctggaactacaccttctggcagtggtttgacggggtgatggaggtgttgaagaagcaccacaagccccactggaatgatgggtaaggaacgggggctgcagggtcaggggccagctgtgggcgcagagagactgtggctgtggcccagtggtgacgctcaatgctccgtgcacccagggccatcctaggttttgtgaataagcaacaggcccacgacctgctcatcaacaagcccgacgggaccttcttgttgcgctttagtgactcagaaatcgggggcatcaccatcgcctggaagtttgactcccgtgagtgcccgttttgcccacactccagccccaaggcccggtctcttgttcccttgccccgccaccccaccctccatcgggcctgtgtccttagaaggtacccagcgggaagcttagtatgagagggctgtggcttggaaatgtattctctttctattgttttccattttggagaacctgaagtccccagccccatagactccaggacggctgggcgagtcctcctgcagtttcacgctctcccctctctgccacgacccagccttgttggtatctgctcatctggtttgctgagtagaacatcccgcatcggctttcttccctacaggctggggctgcagcgcaagctactatataaaagcccagatttctcttgcaagcctgccctaaagccccacaaccttggtcctcctgctgctggtggattatgggaatgaggctgttcttttcacagcggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctgggtgggtactgccccaggaccctgccggctgactcccccgggctcttccccagcccatagacaaagccttgggctgcgccgtggggacttccccaggaggagcctaggggccatgtcccctgtgggttttggcccatggggtggtggagaggatttcagggctcacaaatgaggagagggaacgagaaacccgtcccagctctcttctctgcaaagtgaaagctgcatggattctcacttctgcaccctcggaggaaggggtggcagcactgtagggagtggccgggatcattcgagcccacagatagtgctccgctccccacctcaccagctgctctccacaccctgctcacaaaccttcccccgagtggagtgcaggcagcaaaagggagaagtctctcttcttccagctgccccaaatccattggttgggtttgcttgttgattctcattctttgacaggggtgggagcagggagagggaaatcagatggccagaaaaagaaccagaaggaatgggattcaagccaggggtctcagtgaccctcaggcaggattcatcagctggtgtttattgggggtccttgggaaatctcatcccagctgagaacacaaggtgatgtgagcaggagggagactacatggggcgtgggcttccaccccacttgggagttcccagagactttggttctcaccactgttcttcccttgacagctaaagctgttgatggatatgtgaaaccacagatcaagcaagtggtccctgagtaagtgtccaggtggctgtggctctccttctgcctctttcctcctcccccagaccctgcctcccatcctgatcctgggcccagctttccgctcccccagggaacctgtctcagccctggggagggagtcccctctcctgtagctggagccccagggccgagtggggcactttgtccgtctacaccccgagaaagacaaacatgcccctcggtcccctccccaagtcagctgccccggcctccctggtggctgaagctctgttctcttccttctgcaggtttgtgaatgcatctgcagatgctgggggcagcagcgccacgtacatggaccaggccccctccccagctgtgtgcccccaggctccctataacatgtacccacagaagtaggtggtgttctcatggggtccgcaggggaagaactggggacttggccccaggctggactcctggagggctggcgggcaaaggatccagagctctggttaggcccaagtccaggaggagtgagatcagccaggaaaacagagcattttgaagctagacatagcagggtcaagtcctcactctagtactaactgtgtgatcttgggaaaatgattgagcctctctgatgctcaagtttccttatgtgtaaaacggaggcaagaatgcttgcctcatgagaatgggatgagcattaaactgagacagtgtgggtgaagcatttccacagggaggggcttggaagggcttgatacagggtagctatgaatggggtggttgttcttgagtctggtgggaacaaggtagataattaattcccttcctcactgtggatagccagggctggatgtctgggaggtaccaccttccagtgggaccactgaagatcccaggttccaatccggactccaccacctcccagccaggtggcccagagcaagcttcctgatatggccttggttttctcgtctataaaacggggtcaatacccactctccagttggctgtaaaaattggagtcatgtgctcaataggctcagctgttctaggtgctcagtgaaaagattcagctgttctaagtgctcagtgaacggggcggtgcctaccattgcagctgagcatcattgcaggtgggcagagcccactgtgtcagccacctggttttaagatgaggacacggagaggggaaatgaattccctaagattattcagtgagttagcaagagatgcgaatagagtgggggtctgcagaagccagtgggctgactgctacccagcttggggtggggtgggggcatccagccagaactggtcctgttctcatgagacgggtttgagtggagagcaggctggaggctgtccccagggagcttgaggctgtgagacatgccagctccctctgacatccccctgtctttaccagccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggctgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcat

SEQ ID NO: 2 - DNA, 4298 bp, Homo sapiens
  misc_feature (1)..(4298): cDNA of STAT5A mRNA (from Genbank Accession No. NM_003152.2..gi21618341)
cagacaggatattcactgctgtggcaaggcctgtagagagtttcgaagttaggaggactcaagacggtccctccctggacttttctgaaggggctcaaaagatgacacgcgccagagctggaaggcgtcgccaattggtccaacttttccctcctccctttttgcggatgagaaaaactgaggcccaggtttgggatttccagagcccgggatttcccggcaacgccgacaaccacattcccccggctattctgacccgccccggttccgggacgctccctgggagccgccgccgagggcctgctgggactcccggggaccccgccgtcggggcagcccccacgcccggcgccgcccgccggaacggcgccgctgttgcgcacttgcaggggagccggcgactgagggcgaggcagggagggagcaagcggggctgggagggctgctggcgcgggctcgccggctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggttgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctgggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcat

SEQ ID NO: 3 - protein, 794 aa, Homo sapiens
  MISC_FEATURE (1)..(794): STAT5A protein (from Genbank Accession No. NP_003143.2..gi21618342)
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 4 - DNA, 1569 bp, Homo sapiens
  misc_feature (1)..(1569): cDNA from SMN2 encoding SMN-del7 (from Genbank Accession No.NM_022875.1..gi13259526)
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactcttttgaaaaaccatctgtaaaagactgaggtgggggtgggaggccagcacggtggtgaggcagttgagaaaatttgaatgtggattagattttgaatgatattggataattattggtaattttatggcctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaa

SEQ ID NO: 5 - protein, 282 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMEMLA

SEQ ID NO: 6 - DNA, 1623 bp, Homo sapiens
  misc_feature (1)..(1623): cDNA from SMN2 encoding complete SMN (from Genbank Accession No.NM_017411.2..gi13259525)
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactcttttgaaaaaccatctgtaaaagactgaggtgggggtgggaggccagcacggtggtgaggcagttgagaaaatttgaatgtggattagattttgaatgatattggataattattggtaattttatggcctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaa

SEQ ID NO: 7 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 8 - DNA, 4816 bp, Homo sapiens
  misc_feature (1)..(4816): Genomic region spanning the SMN2 promoter
gatctcggctcactgcaacctccgcctccagggtcaagtgattctccagcctcagcctcctgaatagctgggattacaggcgcccgccaccacgcccagctaatttttgtatttttagtagagatggggtggggtttcaccatgttggccaggctggtcttgaactcctgacatcaagtgttctgctcacctcagcctctgaaagtgctgggattacaggcgtgagccactgcgcccggcagtctttccttcttttttttttttttttttttttttttaatgacatggggtcttactttattactcaggctggtctcaaacttctggcctcaaggaatcttcccaccttggcctcccaaattgctgggattacaggcataagtcatcatgcctggctacaaacagatattttcaataagaggataaaagttcatttccccatactttgctaacatcaaatgttattaattcctaatagttttgccaaactgagaggaaaatggtatgttagtttttctgggttttctttctttttaattttttttcttttttattcatcgcaacactattcacgatttttttattttttattttatttatttatttatttttttttgagacaaggtctccctatgttgcccaggctggtcttgtacccctgggctcaaaggatcctcctgcctcagcctcccaaagtgctaggattacaggcatgagtcaccacgcctggttcacaatttctttttgtttttaccaaaggcaggtatattcctgaaattttttgtttttttgttttttttttgagatgaagtctcaccctgtcactcagactggagtgcactggcacgatctcagctcactgcaacctccgcttcctaggttcaagcgattctcctgcctcagtcttcaaagtagctaggattataggcgccgcaaccatgctcagctaatttttgtatttttagtagagacaggatttcaccatgttggccagggtgatctcaaatcctgacctcaagtgatccgcctgcctcagcctcccaaagtgctgggattactggcatgagccaccgtgccaggccctgaaatgttatcttagttattaatttgcaattccttggctctagaggttgggcatcttctcagatctctagtggacatttggattttctttttggtgaactgtccagtttttctctctgctttacaatctttattatatgcaatcttcacatgtaggtactaccatttttttagtttgtttttgaaacagcatattgctctgttgcccaggctggagcacggtggcaaaaacatggcttactgcagcctttgacctccttggctcaagtactcctcctgtctcagcctcctgagtagctggtaccacaagcccataccaccatgcccagctaatttatttttgtagagatggggcctgaccatgttacttgggctcaaatgatcctctcccactcagcctcccaaagtgctaggattacaggcatgagccaccatacttggcccttttttttttttttttttttttttttttgagacagagtcttgctctgtctcacaggctggtgtgcagtggcacgatctcagctcattgcaacctccacctcccagtttcaagtgatttttgtgcctcagcctccctagtagctgagattacaggcatgcaccaccatgcctggctgactttcatatctttagtgttgccatgttggctaggctggtctcaaactcctgacctcatgtgatccacctgcctcggactcccacagtgctgggattacaggtgttagccaccaccccgacattatttgaaacttttattttatcatgagagagttccaggagtcaactgaagagagatttttggtatgaaaattacatatgcaaaaagactgattccagtacatgaaattaaattcaacatttacattaaatgccttcaaatatggtaaaatggtttcttttggcagtttacctcattatgttttgaatgatttgtctatcatatgaaataacttttataaatatagtaactcaggcctgggcacagcggctcaagtgggaggactgcttaagcaaccgagtttgagaccagcgtggacaacatggggagaccccttctctcccaaaaaatagctgagcatggcagcgcactgctaaaggaaacagagtttctttggtgggtgattaaaatgttctggagttagatagtagtgatggttgcacaaccttgtgaatatattaaggtttccgctctatctaccattcaattgtactctctaaaacgattaattctatagtatatcaattatatctctaaataataaaaacaaaaagaaatggctgggtgcggtggctcatgcctgtaatcgcagcactttgggaggctgaggcgggcggatcacaaggtcaggagtttgagaccaacctgaccaacatggcaaaacccagtctctactaaaaatacaaaaattagctgggtgtggtggcacacgcctgtaatcctaggtactcgggaagctgagacaggagaatcacttgaacccgggaggcagaggttgcagtgagccactgcactccagtctgggtggcagagcgagactccgtctcaaaaagaaaaaaattaaaaaacaaaaagaaacctggttctatattttgtttaaatttatttttttaaccatcatgtaatatgtccaggtaatttgtttaaattttgacatcaaatgcaattgtgagaatttttatgattcagaaaaatctaagcaagctttataaaaacatacttttttttttacttttttttttttttctgagacacagcctcactctgtcgcccaggctggagtgcaggttttcatgtttatctgtgagatgtacctttggcacattactttcctgacatgagatttaaatttttttttttatcttgtgacaatttaacttttttgacacataaaaattgtacatatttatttgtttgagatggagtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgatttttgtatttttaggagaaacagggtttcaccatgttggccaggctggtcttgaagtcctgacctcaagtgatccacccaccttggcctcccaaagtgctgggattataggcatgagccaccgtaccagacccctaaaaattgtatatatttaaggtgtaccatttgatgtttagatatacattgtgaaatgattacattccacatattacctctacagagttaccatttttgtacacttggtcaacatcatcccattctccccttcctccacagatatttcttgtatactatatagaagccaagggtattttgggggaagagctcaaagttcctttcgtggagttaaaaatatatatatactatgtacatataagccatttagcaaccctagatgcttaataaagaatactggaggcccggtgtggtggctcacacctgtaatcccagcactttgggaggccgaggcggtcggattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatactctgtcgcaaaaaaaaaaaagaatactggaggctgggcgaggtggctcacacctgtaatcccagcattttgggatgccagaggcgggcggaatatcttgagctcaggagttcgagaccagcctacacaatatgctccaaacgccgcctctacaaaacatacagaaactagccgggtgtggtggcgtgcccctgtggtcctagctacttgggaggttgaggcgggaggatcgcttgagctcgggaggtcgaggctgcaatgagccgagatggtgccactgcactctgacgacagagcgagactccgtctcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgctatg

SEQ ID NO: 9 - DNA, 6762 bp, Homo sapiens
  misc_feature (1)..(6762): Genomic DNA sequence of the stathmin STMN1 gene (from Genbank Accession No. NC_000001.9..gi89161185)
aggggcactgctctgtccgagtgctgcccttggggcgaggcgggcatgtggctctacaaggtggagtccaggcggccaaagtttggaaaggtagggaaggacccccccgccctccgcctgctccgccctgcccttgttctcgagaatggggagctggttcggacctagtccggggtccactgccacgccctcttccacggcgagaccacccccctagtcccaggcccacacctggggatgctccccaggcgccctgcaacccccggcattgtcctcctgccctccgggacaggactacacttcccgaggtgcttcggggtcccggggggcggcgctccacgcgggttgtggggggcgggggcggcacgtgccgccgctctcggccaatgcggagccccgcgcggaggtcacgtgcctctgtttggcgcttttgtgcgcgcccgggtctgttggtgctcagagtgtggtcaggcggctcggactgagcaggtgggtgcggggctcggaggaggcggcggctggctgaggccagcaagagggacgcggtcggcgggaggggctgggccgtggcagcgaccccctgctgcagggcggcgggcggggctgcgggcctcggaggggttggtgggcgggggtcgctccgctttgtgtgtggctcgggcggagcctcgcctttgtccccgctctccgggggcgcggctgttcgtgggcagggggctgggcgatcaccgggcgtccgctccggggtgccgtcgaggagacaatagggggcgtgggccctcgtttacctccctccctccctcccttccctgcgggccccgccgggttccccattgtctgaagggacggggcggtgccccagggaccagcggctttaggaccaaactgcgggcagccagggccgcgaccctccctgcgaccgtcccctggcgaccgcagctggtgattgaggggcggcgctcccgggccccacgagggttcttctgtcttcgcggccggacgcgcggacagcgtgggtggcggcaggttgggcatggggacggcgggaggcggtggcgagctcaccgcgggaccacccgggggcctgttcccggggcctgccccacccgctgaactgtgaagggggtggtggcggcggcctggaggtgtttttggcgggagttggggggggcgtccgcgcagggggagtcaggcaggggcggagttacccggattggaccgttagccccgcccacccctccccttcccacgcgcgcgggctccggggtgttgagttcggggagattcgaaaaggcgcggggaggaagggggcggggccaggggccggagcgcaaggcgtgctctgattggccgggggcgaccggtcctcttttcctcgcccggaccagggccacgcccatcctgggtccggtgctgcgtctaattctctgcttttcttaaatcttgtcgctgcctctgattttaattcctagcttttgggaacctgtcatcctacgtttttggtactagctggcgtctacaaaagtcataatgttaaaaagatcaacaagagatacagcatttttcatgacataacggcagcaaatataagtcaaaatctagaggttcataaacattttgcttgctgttgggcaaggaagcttaaacctgagggacaataggagttcaacattattggttactattagcttgggcgttttcttatccaccacgtcagacacagacaaagcaggggtgggtatttcatttgcacaatgagttgtaggcagtattaagatggctccgggggcactgttgagttgaatctggaatatcttcttacagtttcggtgaaatgttaaagagtttatgggggaaaaattcttcacccttgtgactttgtctgattttaaaaatccaagagttttatgaccgagaaagctcagttaacttgattttctggaaccaatatcatattcaggtcatatttcccaatgtttatttagtagattttgataatttttttcgtggttaatttagacgtctttattccacgtatttttctgacgatgtatgtagatgtgatgtgagatttttttgggttgatgacatatagaaaggcaaagaaagtgattgcatgtttttgaaaatcattttcaggactttccttatcccagttgattgtgcagaatacactgcctgtcgcttgtcttctattcaccatggcttcttctggtaggtaatctatttggaaaatctgaaattgtaatgggcttatgattttagattgagatggctcaggtcttcgcctttgatttggcacttatgttttggtcttaccaaaacctattttatgaataggagaagaatttaaaaatgattatcacttgaatgtgccgagagctcgtaattgtttattggacagtttggcttagtctgaagcaaaattgtggagtttgcacaagtcttttgtttatgaaagcgattgtcagatactgatgtctcaaaacagtatttattaatccaaaaatgttgagctttgtttttctgggagatggtttttattttttttgagacagggtctcaccttgttgcccaggctggagtgcagtggcttaattatagctcattgcagccttgacttctggagctcaagtgggctcaagcgattctcccacctcagcctccatagcatctgggactatcgacatgggccaccacacccacctaatcaaaaaaaattttttttgtagagatgggctttccctttgttgcccaggctggtctcaaacttcagggctcaagggatcttcccatgttggcctcccacggtgctgggattataggcatgagccatggtacctggccttgggaaatggtatttagataataatatcttgcctgcaaatacatcttcccctagtgtcagtagactgataggaataaaaaggggaaaaaaaacacaactttcctcatcagccctagtttaatacattaaattgatttgggttttagaaaattatagtacagtttattagaacaggagaatcctggttttctgaattataaatataatcaattctagatatccaggtgaaagaactggagaagcgtgcctcaggccaggcttttgagctgattctcagccctcggtcaaaagaatctgttccagaattccccctttcccctccaaagaagaaggatctttccctggaggaaattcagaagaaattagaagctgcagaagaaagacgcaaggtaaacgaagcaattcacagaaagcaggatattaatttatgtaatgggcagatcaattttatttctataacaggaagaaaacagaattgtagctacactgtgattattacatatgccagtgactggaaggaaataccagtcctcatttattgaactcctgttacatgcccgctcctttgtttatatttttctcctttaatacatggtgttgcctcaaataatggaaattagaaacagtttcaggaatgttaagtcgtttttctgcagtcatacaactagtaagtgttggggtcagaattcaagccctggtctatcttaaaccaaagctcatgcttctctcatgcttcctctttgaaaagatttgttgccagatgattctttggcactttggttttgttttttgagagctgtacaataacattttaaattgctagtgtgattgtgttgctcagctggtatcatggtagcttttctcttatctaaacaattctattataaagtaactatctttaaaagctaatcagaagaatcaataaatattaatatgctagttgtagaaaatttgggaaatacagaaatttataaatggaaattaaaagtattcattatcccgcttccgagaagcaaccagtgttaacattttggtgtgtttctttccattcaatgttactcattaacaactgtacataacttttcatttaactccctttctcaacaaccctgataggattgattttaaagctggggcaagtgaggcacaaaaggtaaggtaataaccttccccagaccagcatagtgatttgtagtatacacacacacccgcccgaagagcctcagtgcttacactaaggagtcgtcttctatgcaatagtgtgagttcatggagtaggaggaagcaatacaaccaaaggttggacagtggaaagctttttagacatcaaccctggccctgtagtcattagcctgtgctttacatagtaactggctaaatataatgaaactcccatcatgactaggatttggcagaagagaatcaatagaaccagtgtcagatgctctgtggttatcctgcaagtcagtggttccaatgtgttttgagaacaagctgttctgttgaaggggtcataccaaggtatggtctggtaattaatgcagtttcctgagacaaaagctaataagccttttccttgaaacaaatttttctgtcttaaatagtaatctacagacttagtcttgaatttcctatcattgttttatcagttatggttaaaatttttacaatgagctagttttctttgggtagcttttgaagttaaatagtgaaattctttacaataaaagtgccactcgcaagtacatattcctcaagtcatccagataccattaagcagtaaatcttacaaggatttccttaaggactaattgggtaagatttctgaacagataagcacttttccaaagttaaatacaaatactagaaaaagaatatcattttcacagtattttatgatagggataattcaggtcctaattttggtgttatttaaagggaccttattttctgccccttttcaatccccttagtaaattatttttattgtaattttaacttatgctgaatacattttattttttgaggcagagtctctgtcacccagggtggagtgcagtggcacgatctcagttcactgcaacctccacctcccgggttcaagcaattcttctgcctcagcctcccaagtaacttagactacaggcacccgccaccacgcccggctaatttttgtatttttagtataggtaggatttcaccatgttggccaggctggtcttgaactcctgacctcaaatgatccacccaccttggcctcccaaagtgctgggattacaggcgtgagccaccacacccagcctgaatacattttagagtgcctagcctattaaacttttttttccagtcccatgaagctgaggtcttgaagcagctggctgagaaacgagagcacgagaaagaagtgcttcagaaggcaatagaagagaacaacaacttcagtaaaatggcagaagagaaactgacccacaaaatggaagctaataaagagaaccgagaggcacaaatggctgccaaactggaacgtttgcgagagaaggttggtttcttactttgtaaaagggttgagcttggagtttgatgcaccaatgagttggcttgaactaagtgctttgataaaaggtgtttggtgtctttttgtcatccattttggggcttaacatattaaatgaaaggtatattttaagatggaatattcagtaattcccagcataattgcacagtccttggaagtccagtaggcagcttgttaggttctacaagggacccaggagatttgatgatgatgtctcagaacttaaattgtgtggttcccacaggctgtaatatatgcactgaggttgtgttgggcctctttgaggtgggggctgggggtcgtgacttgacaggcttttttttttttttttttttttgactgatgacaccttacccttcctttacaggataagcacattgaagaagtgcggaagaacaaagaatccaaagaccctgctgacgagactgaagctgactaatttgttctgagaactgactttctccccatccccttcctaaatatccaaagactgtactggccagtgtcattttattttttccctcctgacaaatattttagaagctaatgtaggactgtataggtagatccagatccagactgtaagatgttgttttaggggctaaaggggagaaactgaaagtgttttactctttttctaaagtgttggtctttctaatgtagctatttttcttgttgcatcttttctacttcagtacacttggtgtactgggttaatggctagtactgtattggctctgtgaaaacatatttgtgaaaagagtatgtagtggcttcttttgaactgttagatgctgaatatctgttcacttttcaatcccaattctgtcccaatcttaccagatgctactggacttgaatggttaataaaactgcacagtgctgttggtggcagtgacttcttttgagttaggttaataaatcaagccatagagcccctcctggttgatacttgttccagatggggcctttggggctggtagaaatacccaacgcacaaatgaccgcacgttctctgccccgtttcttgccccagtgtggtttgcattgtctccttccacaatgactgctttgtttggatgcctcagcccaggtcagctgttactttctttcagatgtttatttgcaaacaaccattttttgttctgtgtcccttttaaaaggcagattaaaagcacaagcgtgtttctagagaacagttgagagagaatctcaagattctacttggtggtttgcttgctctacgttacaggtggggcatgtcctcatcctttcctgccataaaagctatgacacgagaatcagaatattaataaaactttatgtactgctgtagcaa

SEQ ID NO: 10 - DNA, 1730 bp, Homo sapiens
  misc_feature (1)..(1730): cDNA of Stathmin STMN1 mRNA (longest transcript variant from Genbank Accession No. NM_203401.1.. gi44890051; also see variants 2 and 3 thereof)
atcaccgggcgtccgctccggggtgccgtcgaggagacaatagggggcgtgggccctcgtttacctccctccctccctcccttccctgcgggccccgccgggttccccattgtctgaagggacggggcggtgccccagggaccagcggctttaggaccaaactgcgggcagccagggccgcgaccctccctgcgaccgtcccctggcgaccgcagctggtgattgaggggcggcgctcccgggccccacgagggttcttctgtcttcgcggccggacgcgcggacagcgtgggtggcggcaggactttccttatcccagttgattgtgcagaatacactgcctgtcgcttgtcttctattcaccatggcttcttctgatatccaggtgaaagaactggagaagcgtgcctcaggccaggcttttgagctgattctcagccctcggtcaaaagaatctgttccagaattccccctttcccctccaaagaagaaggatctttccctggaggaaattcagaagaaattagaagctgcagaagaaagacgcaagtcccatgaagctgaggtcttgaagcagctggctgagaaacgagagcacgagaaagaagtgcttcagaaggcaatagaagagaacaacaacttcagtaaaatggcagaagagaaactgacccacaaaatggaagctaataaagagaaccgagaggcacaaatggctgccaaactggaacgtttgcgagagaaggataagcacattgaagaagtgcggaagaacaaagaatccaaagaccctgctgacgagactgaagctgactaatttgttctgagaactgactttctccccatccccttcctaaatatccaaagactgtactggccagtgtcattttattttttccctcctgacaaatattttagaagctaatgtaggactgtataggtagatccagatccagactgtaagatgttgttttaggggctaaaggggagaaactgaaagtgttttactctttttctaaagtgttggtctttctaatgtagctatttttcttgttgcatcttttctacttcagtacacttggtgtactgggttaatggctagtactgtattggctctgtgaaaacatatttgtgaaaagagtatgtagtggcttcttttgaactgttagatgctgaatatctgttcacttttcaatcccaattctgtcccaatcttaccagatgctactggacttgaatggttaataaaactgcacagtgctgttggtggcagtgacttcttttgagttaggttaataaatcaagccatagagcccctcctggttgatacttgttccagatggggcctttggggctggtagaaatacccaacgcacaaatgaccgcacgttctctgccccgtttcttgccccagtgtggtttgcattgtctccttccacaatgactgctttgtttggatgcctcagcccaggtcagctgttactttctttcagatgtttatttgcaaacaaccattttttgttctgtgtcccttttaaaaggcagattaaaagcacaagcgtgtttctagagaacagttgagagagaatctcaagattctacttggtggtttgcttgctctacgttacaggtggggcatgtcctcatcctttcctgccataaaagctatgacacgagaatcagaatattaataaaactttatgtactgctgtagcaaaaaaaaaaaaaaaaaa

SEQ ID NO: 11 - protein, 149 aa, Homo sapiens
  MISC_FEATURE (1)..(149): Stathmin STMN1 protein
MASSDIQVKELEKRASGQAFELILSPRSKESVPEFPLSPPKKKDLSLEEIQKKLEAAEERRKSHEAEVLKQLAEKREHEKEVLQKAIEENNNFSKMAEEKLTHKMEANKENREAQMAAKLERLREKDKHIEEVRKNKESKDPADETEAD

SEQ ID NO: 12 - DNA, 21 bp, Artificial
  Stat5A binding probe containing a novel ′ctctactaa′ Stat5A binding site
cccagtctctactaaatacaa

SEQ ID NO: 13 - DNA, 21 bp, Artificial
  Stat5A binding probe containing a consensus ′ttctaggaa′ Stat5A binding site
agatttctaggaattcaatcc

SEQ ID NO: 14 - DNA, 22 bp, Artificial
  Non-specific competitor probe containing an SP1 consensus′ccgccc′ binding site
gctcgccccgccccgatcgaat

SEQ ID NO: 15 - DNA, 21 bp, Artificial
  Stat5A binding probe containing a mutated ′ctttactta′ novelStat5A binding site sequence
cccagtctttacttaatacaa

SEQ ID NO: 16 - DNA, 10 bp, Artificial
  Generic 10nt-long consensus GAS element sequence
ttcnnnngaa

SEQ ID NO: 17 - DNA, 10 bp, Artificial
  Generic 10nt-long non-consensus GAS element sequence 1
ttcnnnngag

SEQ ID NO: 18 - DNA, 10 bp, Artificial
  Generic 10nt-long non-consensus GAS element sequence 2
ttcnnnngta

SEQ ID NO: 19 - DNA, 18 bp, Artificial
  Primer Stat5A1*6 forward
catggcgggctggattca

SEQ ID NO: 20 - DNA, 18 bp, Artificial
  Primer Stat5A1*6 backward
tcaggacagggagcttct

SEQ ID NO: 21 - DNA, 20 bp, Artificial
  Primer SMN forward
tgtagtggaaagttggggac

SEQ ID NO: 22 - DNA, 23 bp, Artificial
  Primer SMN backward
cctggcattgggggtggtggagg

SEQ ID NO: 23 - DNA, 20 bp, Artificial
  Primer Gapdh forward
cccttcattgacctcaacta

SEQ ID NO: 24 - DNA, 20 bp, Artificial
  Primer Gapdh backward
ccaaagttgtcatggatgac

SEQ ID NO: 25 - DNA, 24 bp, Artificial
  Primer b-Actin forward
atggtgggaatgggtcagaaggac

SEQ ID NO: 26 - DNA, 24 bp, Artificial
  Primer b-Actin backward
ctctttgatgtcacgcacgatttc

SEQ ID NO: 27 - DNA, 24 bp, Artificial
  Primer Exon2a forward
ctgacatttgggatgatacagcac

SEQ ID NO: 28 - DNA, 23 bp, Artificial
  Primer Exon6 backward
tggtggagggagaaaagagttcc

(19) United States
(12) Patent Application Publication
(10) Pub. No.: US 2008/0187512 A1
(43) Pub. Date: Aug. 7, 2008

(54) Treatment for spinal muscular atrophy

(75) Inventor: Hung Li, Taipei (TW)

Correspondence Address: KING & SPALDING LLP, 1180 PEACHTREE STREET, ATLANTA, GA, 30309-3521, US

(73) Assignee: Academia Sinica, Taipei (TW)

(21) Appl. No.: 11/703,810

(22) Filed: Feb. 7, 2007

(51) Int. Cl. A61K 38/20; A61K 31/70; A61K 33/00; A61P 21/00; G01N 33/00; C12Q 1/68; A61K 38/00; A61K 38/16

(52) U.S. Cl. 424/85.2; 424/600; 435/6; 436/86; 514/12; 514/44; 514/8; 536/24.3

(57) ABSTRACT

Compositions for treatment of spinal muscular atrophy (SMA) and methods for use thereof to treat SMA and other conditions of SMN-deficiency; novel drug development targets for SMA therapies, and methods of use thereof to screen for candidate therapeutic and diagnostic agents.

### BACKGROUND

The present disclosure relates to spinal muscular atrophy and related genetic disorders, methods for treatment thereof, and drug target sites for development of therapeutic and diagnostic agents therefor.

The statements in this section merely provide background information related to the present disclosure and may not constitute prior art.

Proximal spinal muscular atrophy (SMA) is a class of motor neuron degeneration disorders for which there is currently no effective treatment. Compared to other human autosomal recessive disorders, it is relatively common, occurring in about 1 of every 6000 newborns, and it is the most common hereditary cause of infant mortality.

SMA develops and progresses due to a reduced level of Survival Motor Neuron (SMN) protein that occurs through either homozygous deletion of the SMN1 gene or impairing mutations in all inherited copies of the SMN1 gene. A second SMN-encoding gene, SMN2, also present in the human genome, is nearly identical to the SMN1 gene, but encodes as its main product a defective SMN protein, SMNΔ7, which is not able to compensate for the loss of the SMN1-encoded SMN. The SMN2 gene's defects result in a different splicing of the pre-mRNA SMN transcript so as to exclude exon 7 from the mRNA and introduce a premature stop codon, thereby expressing SMNΔ7. Yet, a small percentage of SMN2 transcripts are correctly spliced in spite of the mutation, resulting in net expression of a low level of functioning SMN.

The class of SMAs resulting from such SMN deficiency includes childhood proximal SMA, X-linked recessively inherited bulbospinal SMA, and distal SMAs such as scapuloperoneal SMA, scapulohumeral SMA, facioscapulohumeral SMA, oculopharyngeal SMA, Ryukyuan SMA, and others. Proximal SMA is subdivided into clinical Types I, II, III, and IV, based on age of onset and severity of symptoms. Thus, a spectrum of SMAs is found, all of which involve low levels of motor neuron SMN.

In addition to the SMAs, a subclass of neurogenic-type arthrogryposis multiplex congenita (congenital AMC) has separately been reported to involve SMN1 gene deletion, suggesting that some degree of pathology in those afflicted is likely due to low levels of motor neuron SMN. L. Burgien et al., Survival motor neuron gene deletion in the arthrogryposis multiplex congenita-spinal muscular atrophy association, J. Clin. Invest. 98(5):1130-32 (September 1996). Congenital AMC affects humans and animals, e.g., horses, cattle, sheep, goats, pigs, dogs, and cats. See, e.g., M. Longeri et al., Survival motor neuron (SMN) polymorphism in relation to congenital arthrogryposis in two Piedmont calves (piemontese), Genet. Sel. Evol. 35:S167-S175 (2003). Also, the risk of development or the severity of amyotrophic lateral sclerosis (AMLS) has been found to be correlated with low levels of motor neuron SMN.

Therefore, it would be advantageous to provide novel methods for increasing motor neuron SMN levels in order to treat those afflicted with SMA, with neurogenic congenital AMC, or with other SMN-deficiency-related conditions. It would further be advantageous to provide novel drug targets that could be used as a basis for developing effective therapeutics or diagnostics for such neuronal conditions.

### SUMMARY

In various embodiments, the present technology provides novel methods for increasing motor neuron SMN levels so as to treat subjects having spinal muscular atrophy or another SMN-deficiency condition.

Various embodiments of the present invention further provide: Methods for treating spinal muscular atrophy (SMA) or other SMN-deficiency in a subject, involving administering a therapeutically effective amount of a pharmaceutically acceptable activator of Stat5;

Methods for treating SMA or other SMN-deficiency in a subject, involving administering a recombinant genetic vector containing at least one copy of a host-expressible gene encoding Stat5A; such methods in which the Stat5A is a constitutively activated Stat5A, such as, e.g., Stat5A1*6; such methods in which the vector is a viral vector, such as, e.g., an adenoviral, adeno-associated viral, herpes viral, or lentiviral vector;

Methods for identifying a candidate compound for treatment of SMA, involving (1) contacting a test compound, under Stat5-activation-permissible conditions, with a Stat5(+) mammalian cell that contains an expressible, Stat5-activatable target nucleic acid whose promoter contains at least one Gamma-Activated Sequence (GAS) element and at least one CTCNNNTAA motif, and (2) detecting the level of expression of the target nucleic acid or of a phenotypic effect resulting from expression thereof, wherein (3) an increased level identifies the test compound as a candidate compound; such methods in which the cell is Stat5(+)/SMN2(+) and the detection involves assaying the level of SMN2 transcripts, the level of SMN or SMNΔ7 protein, or the occurrence of nuclear gems in the cell nucleus;

Nucleobase probes containing a base sequence of CTCNNNTAA or the complement thereof, or the RNA base equivalent to either of these;

Methods for identifying a candidate Stat5-regulated gene or promoter thereof, involving (1) contacting such a nucleobase probe, under specific-hybridization-permissible conditions, with a gene-containing cell, cell fragment, or polynucleotide preparation, (2) removing non-specifically-hybridized probes, and (3) detecting remaining hybrids and determining that the target sequence to which the base sequence of the probe has bound is located in a gene promoter region, wherein (4) such detection and determination identifies the promoter's gene as a candidate Stat5-regulated gene, and/or the promoter as a Stat5-related promoter;

Methods for identifying a candidate Stat5 protein, involving (1) contacting such a nucleobase probe, under specific-protein-binding-permissible conditions, with a polypeptide having the amino acid sequence of Stat(5) or an amino acid sequence at least 70% identical thereto, (2) removing non-specifically-bound probes, and (3) detecting remaining polypeptide-probe complexes, wherein (4) such identifies the polypeptide thereof as a candidate Stat5 protein;

Methods for treating SMA or other SMN-deficiency in a subject, involving administering to the subject a recombinant genetic vector that contains at least one copy of a host-expressible gene encoding Stat5A and at least one copy of a Stathmin inhibitor; such methods in which the Stat5A is a constitutively activated Stat5A, such as, e.g., Stat5A1*6; such methods in which the inhibitor in a Stathmin expression inhibitor, e.g., an RNAi nucleic acid, such as an shRNA; and such methods in which the vector is a viral vector, such as, e.g., an adenoviral, adeno-associated viral, herpes viral, or lentiviral vector.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The patent or application file contains at least one drawing executed in color. Copies of this patent or patent application publication with color drawing(s) will be provided by the USPTO upon request and payment of the necessary fee.

FIGS. 1A-G present gel images and control, 4, 8, and 12 h bar charts of semi-quantitative RT-PCR analysis of the effects of sodium vanadate, trichostatin A (TSA), and aclarubicin on cellular production of SMNΔ7 and full-length-SMN transcripts from SMN2. (A) Image of gel used for selection of SMA-afflicted mouse embryos used as source of cells for (B-G); and results for: (B) SMA-like MEF cells treated with 50 μM sodium vanadate, (E) SMN2-NSC34 cells treated with 100 μM of sodium vanadate, (C, F, respectively) SMA-like MEF and SMN2-NSC34 cells treated with 10 nM TSA, and (D, G, respectively) SMA-like MEF and SMN2-NSC34 cells treated with 80 nM aclarubicin. C_{H2O} or C_{70 % alc}=control cells treated with H₂O or 70% ethanol. Asterisks are: *, P<0.05, **, P<0.005 and ***, P<0.001, by t-test, using at least triplicate results.

FIG. 2 presents a bar chart analysis of the effect of Stat5A expression on SMN protein expression in SMN2-NSC34 cells transiently transfected with increasing amounts (1-4 μg) of a Stat5A1*6 construct. Transfections were repeated at least 3 times and an anti-human SMN antibody was used to Western blot for expressions levels. α-Tubulin was used as internal control, and mean±SEM was calculated. Asterisks: *, P=0.047 and * P=0.0026, versus vector-only control by t-test.

FIGS. 3A-D present graphs of competitive binding assay results for Stat5A binding to a novel binding site motif (CTCNNNTAA) identified in the SMN2 promoter. Competition is shown with unlabeled probes of (A) the novel sequence; (B) a Stat5A-specific binding site sequence previously recognized as a Stat5A consensus binding site sequence; (C) a mutated version of the novel binding site sequence; and (D) a SP1-specific binding site sequence as a non-specific competitor. From triplicate experiments, mean±SEM was calculated. Asterisks are: **, P<0.01 and ***, P<0.001, compared with competitor-free group, by t-test.

FIGS. 4A-J presents results of nuclear staining of Type I SMA-afflicted patients' cells with (G-J) and without (D-F) Stat5A1*6 transfection, and of normal cells (A-C), for the presence of SMN and nuclear gems; FIG. 4K presents results of Western blots (K) for SMN protein expression in these three; and FIGS. 4L-M present results of gem detection assays therein.

FIGS. 5A-B presents images of stained nuclei showing that Stat5A expression enhances neurite outgrowth in SMA motor neurons; (A) morphology of Smn−/−, SMN2, V5-SMN cells; (B) morphology of Smn−/−, SMN2, Stat5A1*6 cells. FIGS. 5C-D present bar charts quantifying axon outgrowth resulting therefrom.

FIG. 6 presents a partial ribbon diagram of the STAT5A dimer, showing domain 2 (blue), domain 3a (red), domain 3b (green), and domain 4 (yellow); taken from FIG. 1 of D. Neculai et al., “Structure of the unphosphorylated STAT5a dimer,” J. Biol. Chem. 280(49):40782-787 (Dec. 9, 2005).

It should be noted that the figures set forth herein are intended to exemplify the general characteristics of an apparatus, materials and methods among those of this technology, for the purpose of the description of such embodiments herein. These figures may not precisely reflect the characteristics of any given embodiment, and are not necessarily intended to define or limit specific embodiments within the scope of this technology.

### BRIEF DESCRIPTION OF SEQUENCES

Sequences are presented in the accompanying Sequence Listing as shown in Table 1.

TABLE 1
Sequences Listed

| SID | Description |
|---|---|
| 1 | genomic DNA sequence of the Stat5A gene; |
| 2 | DNA sequence of the Stat5A cDNA; |
| 3 | amino acid sequence of the Stat5A protein; |
| 4 | DNA sequence of the SMN2Δ7 cDNA; |
| 5 | amino acid sequence of the SMNΔ7 protein; |
| 6 | DNA sequence of the “complete” SMN2 cDNA; |
| 7 | amino acid sequence of the complete SMN protein; |
| 8 | genomic DNA sequence of the SMN2 gene promoter; |
| 9 | genomic DNA sequence of the Stathmin STMN1 gene; |
| 10 | DNA sequence of the Stathmin STMN1 cDNA; |
| 11 | amino acid sequence of the Stathmin STMN1 protein; |
| 12 | DNA sequence of a novel Stat5A binding site probe; |
| 13 | DNA sequence of a consensus Stat5A binding site probe; |
| 14 | DNA sequence of a consensus SP1 binding site probe; |
| 15 | DNA sequence of a mutated Stat5A binding site probe; |
| 16 | DNA sequence of a 10 nt-long consensus GAS element; |
| 17 | DNA sequence of a first 10 nt-long non-consensus GAS element; |
| 18 | DNA sequence of a second 10 nt-long non-consensus GAS |
| | element. |
| 19 | DNA sequence of primer Stat5A1*6 forward |
| 20 | DNA sequence of primer Stat5A1*6 backward |
| 21 | DNA sequence of primer SMN forward |
| 22 | DNA sequence of primer SMN backward |
| 23 | DNA sequence of primer Gapdh forward |
| 24 | DNA sequence of primer Gapdh backward |
| 25 | DNA sequence of primer β-Actin forward |
| 26 | DNA sequence of primer β-Actin backward |
| 27 | DNA sequence of primer Exon2a forward |
| 28 | DNA sequence of primer Exon6 backward |

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Background” and “Summary”) and sub-headings used herein are intended only for general organization of topics within the present disclosure, and are not intended to limit the disclosure of the technology or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, and methods of this technology.

The survival motor neuron (SMN) protein, when expressed at only a low level of functional SMN, has been found to be a key cause of SMA. S. Jablonka et al., The role of SMN in spinal muscular atrophy, J. Neurology 247(13):1432-1459 (March 2000). As a result, as an approach to treating SMA, it is desirable to increase the level of functional SMN expression. Yet, until the present work, the factors directly responsible for expression of SMN have remained unknown. In various embodiments, the present technology provides methods for manipulating Stat5A, a factor found to be directly responsible for expression of SMN, as described in C.-H. Ting et al., Stat5 constitutive activation rescues defects in spinal muscular atrophy, Hum. Molec. Genet. doi:10.1093/hmg/ddl482 (Epubl. Jan. 12, 2007).

### Stat5A

In various embodiments of the present technology, a Stat5A nucleic acid or a Stat5A-activating substance is used in order to increase the expression of SMN2 in a cell, including embodiments for treating SMN-insufficiency conditions, such as a spinal muscular atrophy (SMA). In various embodiments hereof, a Stat5A protein or Stat5A nucleic acid is utilized to screen for candidate substances that may, directly or through a secondary messenger, increase the expression and/or activation level of Stat5A protein, and that can thereby be identified as drugs, or as targets for development of drugs, to treat SMN-insufficiency conditions, such as a spinal muscular atrophy (SMA).

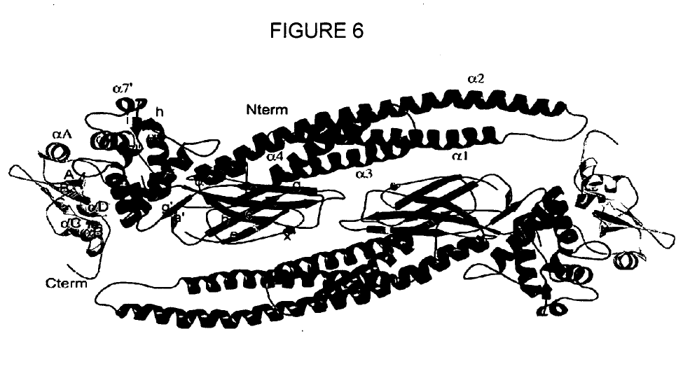

Stat5A is “Signal Transducer and Activator of Transcription” number 5A, a protein that in its native form, upon phosphorylation by a tyrosine kinase, becomes active as an activator of transcription, typically in the form of a homodimer. A variety of cytokines, peptide hormones, and small molecules have been found capable of activating Stat5A, though action of one or more kinases. An exemplary human Stat5A amino acid sequence is presented in SEQ ID NO:3. The structure of Stat5A proteins and genes has been characterized in humans and in animals. See, e.g., R. Ambrosio et al., The structure of human STAT5A and B genes reveals two regions of nearly identical sequence and an alternative tissue specific STAT5B promoter, Gene 285(1-2):311-18 (Feb. 20, 2002). The Stat5A protein contains four domains, described in order from amino-to-carboxy termini as follows, with the numbering of secondary structures for domains 2, 3a/b, and 4 shown according to FIG. 6, and alternative numbering according to E. Soldaini et al., DNA Binding Site Selection of Dimeric and Tetrameric Stat5 Proteins Reveals a Large Repertoire of Divergent Tetrameric Stat5a Binding Sites, Mol. Cell. Biol. 20(1):389-401 (January 2000).

Domain 1 is a STAT protein interaction domain of pfam Accession No. PF02865, which allows Stat5A to dimerize with another Stat5A (or Stat5B) protein so as to become capable of transcriptional activation. Domain 1 is shown in SEQ ID NO:3 as approximately residues 2-122, or alternatively as 2-145. This oligomerization domain comprises a multi-alpha-helix, hook-shaped structure. See, e.g., U. Vinkemeier et al., Structure of the amino-terminal protein interaction domain of STAT-4, Science 279(5353):1048-52 (Feb. 13, 1998).

Domain 2 is a STAT protein all-alpha domain of pfam Accession No. PF01017. Domain 2 is shown in SEQ ID NO:3 as approximately residues 138-330, or alternatively as residues 145-330. This domain comprises a four-helix bundle, α1-α2-α3-α4-, and contains a coiled-coil structure centered at about residue 248 of SEQ ID NO:3. See H. Nakajima et al., Functional interaction of STAT5 and nuclear receptor co-repressor SMRT: implications in negative regulation of STAT5-dependent transcription, EMBO J. 20(23):6836-44 (Dec. 3, 2001).

Domain 3 is a DNA binding domain of pfam Accession No. PF02864. Domain 3 is shown in SEQ ID NO:3 as residues 332-583 and continuing to residue 592. This domain comprises: (a) a DNA-contacting, eight-stranded β-barrel subdomain ‘a’ (approximately residues 331-470, and in an alternative numbering continuing to residue 496), βa-βa′-βb-βc-α4′-βe-βf-βg-βg′-, which is alternatively numbered as the eleven-β-stranded β1-β2-β3-β4-α5-β5-β6-β7-β8-β9-β10-β11-α6-; and (b) an α-helical linker subdomain ‘b’ (approximately residues 471-(or 497)-592), α5-βh-α6-α7-α7′-βi-α7″-α8-, or alternatively α7-α8-βA-α9-α10-α11-.

Domain 4 is a Src homology 2 (SH2) domain of pfam Accession No. PF00017 and NCBI CDD Accession No. cd00173. Domain 4 is shown in SEQ ID NO:3 as approximately residues 593-635, and alternatively continuing to residue 676, or where the remainder of the Stat5A core is attributed to Domain 4, to residue 712. This domain comprises a mixed 4alpha-3beta domain, αA-βA-βB-βC-αB-αC-αD-, or alternatively αA-βB-βC-βD-βD′-αB-αB′-αC-. The region from residues 676-701 is referred to as a “phosphorylation tail segment” and contains residues that become phosphorylated; the region downstream from residue 701 is referred to as a “transactivation” domain and also contains residues that can become phosphorylated.

As a result, Stat5A comprises a secondary structure of: amino-terminus-[alpha Hook]-[α1-α2-α3-α4-]-[(βa-βa′-βb-βc-α4′-βe-βf-βg-βg′)-(α5-βh-α6-α7-α7′-βi -α7″-α8)]-[αA-βA-βB-βC-αB-αC-αD]-[P tail]-[Transactivation Domain]-carboxy terminus. A number of amino acids are also found conserved in this structure, as important sites for activation of the protein itself, as well as for its functioning as an activator of transcription.

Stat5A phosphorylation at Tyr694, as shown in SEQ ID NO:3, activates non-constitutively active (native) forms of Stat5A. Serine phosphorylation is also reported at Ser780 and Ser127/Ser128 of Stat5A, as potentiating its activity in some modes of transcription activation. D. E. Clark et al., ERBB4/HER4 Potentiates STAT5A Transcriptional Activity by Regulating Novel STAT5A Serine Phosphorylation Events, J. Biol. Chem. 280(25):24175-180 (Jun. 24, 2005). Separately, phosphorylation at Ser726 has been reported as enhancing Stat5A activity. I. Beuvink et al., Stat5A Serine Phosphorylation, J. Biol. Chem. 275(14):10247-255 (Apr. 7, 2000). As a result, Tyr694 is strictly conserved in non-constitutively active forms of Stat5A as a site of activation; and Ser127, Ser128, Ser726, and Ser780 are normally conserved as sites of potentiation or activity regulation. In addition, F752-D753-L754 is a conserved tripeptide residue within the alpha-helix structure located at residue positions 752-763 of SEQ ID NO:3. C. M. Litterst et al., NCoA-1/SRC-1 Is an Essential Coactivator of STAT5 That Binds to the FDL Motif in the -Helical Region of the STAT5 Transactivation Domain, J. Biol. Chem. 278(46):45340-351 (Nov. 14, 2003).

Stat5A proteins are very similar in structure to the Stat5B proteins. However, Stat5A proteins are distinguished from Stat5B proteins by, among other features: 1) a conserved Tyr679 in Stat5B, occupying the cognate site of Trp679 of SEQ ID NO:3, which site is occupied by a non-Tyr residue in (native) Stat5A proteins; 2) the insertion of a “C-E-S-A-T” peptide in Stat5B at a cognate position that is between Leu687 and Ala688 of Stat5A; 3) the occurrence of a “Q-W-1-P-H-A-Q-S” C-terminal peptide in Stat5B place of the C-terminal “L-D-S-R-L-S-P-P-A-G-L-F-T-S-A-R-G-S-L-S,” peptide following Ser774 in SEQ ID NO:3; and 4) the native human Stat5B protein is 787 residues long (see Genbank Accession No. NP_{—}036580) as versus the 794 residue sequence of Stat5A (SEQ ID NO:3).

In various embodiments of human-type Stat5A proteins hereof, Stat5A proteins include Stat5A proteins that have amino acid sequences that are at least or about 70%, 75%, 80%, 85%, or 90% identical to that of SEQ ID NO:3, and that retain the conserved primary, secondary, and tertiary structural features and function of Stat5A; and/or Stat5A proteins that have amino acid sequences that are at least or about 75%, 80%, 85%, or 90% similar to that of SEQ ID NO:3, and that retain the conserved primary, secondary, and tertiary structural features and function of Stat5A; based on comparison between aligned sequences. Similarity can be defined with reference to conservative amino acid substitution groups, such as are known in the art; exemplary substitution groups include: Asp, Glu; Asn, Gln; Asn, Asp, Glu, Gln; Ile, Leu, Val; Ile, Leu, Val, Met, Phe; Arg, Lys; Arg, Lys, His; Ala, Gly; Ala, Gly, Pro, Ser, Thr; Ser, Thr; Ser, Thr, Tyr; Phe, Tyr; Phe, Trp, Tyr; non-cystine Cys, Ser; and non-cystine Cys, Ser, Thr. Alignment of sequences can be performed according to any method known useful in the art, such as those described, e.g., in U.S. Pat. No. 7,160,868 to Murphy et al. In some embodiments, identical or similar amino acid sequences can be at least or about 90% or 95% as long as the amino acid sequence of SEQ ID NO:3.

In various embodiments of a human-type Stat5A protein, the amino acid sequence thereof can be at least or about 92%, 93%, 94%, or 95% identical to that of SEQ ID NO:3, or at least or about 94%, 95%, or 96% similar to that of SEQ ID NO:3, based on comparison between aligned sequences.

Stat5A proteins useful in various embodiments hereof include non-constitutively active Stat5A proteins having the conserved Tyr694 activation site, and constitutively active Stat5A mutants. Constitutively active Stat5 mutants useful herein include any constitutively activated Stat5A protein. In some embodiments, a constitutively activated Stat5A1*6 mutant can be used, which is a Stat5A protein that contains H298R and S710F mutations, as described in M. Onishi et al., Identification and characterization of a constitutively active STAT5 mutant that promotes cell proliferation, Mol. Cell. Biol. 18(7):3871-79 (July 1998). Other constitutively activated Stat5 mutants include STAT5A-N642H, which contains a N642H mutation, as described in K. Ariyoshi et al., Constitutive activation of STAT5 by a point mutation in the SH2 domain, J. Biol. Chem. 275(32):24407-13 (Aug. 11, 2000); and STAT5A6-E150G, which contains E150G and S710F mutations, as described in K. Yamada et al., Constitutively active STAT5A and STAT5B in vitro and in vivo, Int'l J. Hematol. 71(1):46-54 (January 2000).

### Stat5A DNA Binding

Stat5A, as a novel activator of SMN gene transcription, binds to the promoter region of the SMN2 gene, and putatively also to the identical-sequence promoter region of the SMN1 gene. B. Boda et al., Survival motor neuron SMN1 and SMN2 gene promoters: identical sequences and differential expression in neurons and non-neuronal cells, Eur. J. Hum. Genet. 12(9):729-37 (September 2004). A number of novel Stat5A DNA binding sites within the SMN2 promoter region have been elucidated herein. See SEQ ID NO:8. These include both IFN-γ-activated sequence (GAS) elements and novel Stat5A binding motifs.

SEQ ID NO:8 sets forth twelve putative non-canonical GAS elements, of which those centered at bases 1862 and 4271 of SEQ ID NO:8 (at positions −2791 and −382 in the promoter region) appear most similar to the canonical ‘ttcynrgaa’ GAS sequence. See E. Soldaini et al., DNA Binding Site Selection of Dimeric and Tetrameric Stat5 Proteins Reveals a Large Repertoire of Divergent Tetrameric Stat5a Binding Sites, Mol. Cell. Biol. 20(1):389-401 (January 2000). SEQ ID NO:8 further indicates three novel Stat5A binding sites, sharing a consensus ‘ctcnnntaa’ motif, centered at bases 946, 2478, and 4407. As a result, manipulation of Stat5A can be employed to cause activation of genes having promoters containing a combination of such GAS element(s) and novel Stat5A binding site(s). Similarly, nucleobase probes comprising such a novel Stat5A binding site can be used to screen for Stat5A proteins.

### Nucleic Acid Constructs

In various embodiments, nucleic acid vectors are useful herein to increase Stat5A transcription/expression levels and/or to introduce nucleic acids encoding enhanced Stat5A proteins such as constitutively active Stat5A proteins. Such vectors can further contain additional genetic factors such as, e.g., those that can enhance the frequency of proper (full-length) SMN2 splicing, increase the copy number of a SMN gene in the target cell, and/or those that can knock-down stathmin expression levels in the target cell.

Stat5A nucleic acids useful herein include any, expressible by a desired host cell, that encodes a Stat5A protein. In various embodiments, the encoded Stat5A protein can be a constitutively active Stat5A protein, such as any of those described above.

SMN2 nucleic acids useful herein include any SMN2 gene and any SMN2-enhancing nucleobase polymers, such as the SMN2 splice-enhancing nucleic acids described in C. Madocsai et al., Correction of SMN2 pre-mRNA splicing by antisense U7 small nuclear RNAs, Mol. Ther. 12(6):1013-22 (December 2005) (Epub Oct. 14, 2005); L. A. Skordis et al., Bifunctional antisense oligonucleotides provide a trans-acting splicing enhancer that stimulates SMN2 gene expression in patient fibroblasts, Proc. Nat'l Acad. Sci. USA 100(7):4114-19 (Apr. 1, 2003); and L. Cartegni & A. R. Krainer, Correction of disease-associated exon skipping by synthetic exon-specific activators, Nature Struct. Biol. 10(2)120-25 (February 2003).

Although the splicing factors described therein are directed to SMN2 exon 7, the methods described can be followed to prepare similar nucleic acid factors that are directed to SMN2 exon 3 and/or exon 5, which exons are also sometimes incorrectly spliced out of SMN2 transcripts.

The present inventors have also separately discovered that elevated stathmin protein levels are directly involved in the motor neuron pathology of SMA. See H. Li, Methods of Diagnosis of Spinal Muscular Atrophy and Treatments Thereof (U.S. patent application Serial No. unassigned; Attorney Docket No. 15069-000003, filed concurrently herewith). Thus, in some embodiments, a stathmin knockdown construct can be included in a nucleic acid vector hereof.

Knockdown refers to introduction into a cell of a nucleobase polymer, such as a nucleic acid, that can decrease the level of expression of a selected target gene; this differs from knock-out or gene silencing techniques that would eliminate target gene expression altogether. Stathmin knockdown can be performed by use of RNAi technology, such as by introducing, into the cell, either (1) a controlled amount of stathmin RNA-targeted siRNA or morpholino oligo molecules, or (2) a host-cell-expressible construct encoding stathmin RNA-targeted shRNA. In various embodiments, nucleic acid from which a stathmin RNA-targeted shRNA can be expressed, can be used for this purpose. For example, MISSION shRNA nucleic acids (knockdown RNAi nucleic acids available from Sigma-Aldrich, Inc., St. Louis, Mo., USA) can be used, according to manufacturer's instructions. The expressible, shRNA-encoding sequence is operably attached to a promoter, e.g., a U6 promoter. The resulting construct is delivered to the cell for nuclear importation and expression.

Sequences useful for preparing stathmin knockdown RNAi nucleic acids can be readily obtained from, e.g., SEQ ID Nos:9 and 10 hereof, and can be prepared according to methods known in the art, such as those described in K. Ui-Tei et al., Guidelines for the selection of highly effective siRNA sequences for mammalian and chick RNA interference, Nucl. Acids Res. 32:936-48 (2004). The shRNA sequences identified can then be included in constructs and delivered, e.g., via vectors, to motor neuron cells. One such method for stathmin knockdown is described in P. Holmfeldt et al., Aneugenic Activity of Op18/Stathmin Is Potentiated by the Somatic Q18 E Mutation in Leukemic Cells, Mol. Biol. Cell. 17(7):2921-2930 (July 2006), which employs an Epstein-Barr viral vector for constitutive expression of stathmin-targeted shRNA. An exemplary stathmin target sequence for use in preparing an RNAi (e.g., shRNA) molecule for stathmin knockdown is CGTTTGCGAGAGAAGGATA (nt728-746 of SEQ ID NO:10).

Commercially available stathmin-targeted shRNA nucleic acids, or the sequences thereof, can be used. Examples of these include SURESILENCING shRNA STMN1 LAP18/Lag Human Stathmin 1/oncoprotein 18 (stathmin-targeting shRNA available from SuperArray Bioscience Corporation, Frederick, Md., USA), and HuSH 29mer shRNA Constructs against STMN1 (Cat. No. TR318815, available from OriGene Technologies, Inc., Rockville, Md., USA).

Knockdown techniques have been developed for therapeutic use in neuron-based disorders. See, e.g., F. P. Manfredsson et al., RNA knockdown as a potential therapeutic strategy in Parkinson's disease, Gene Therap. 13:517-24 (2006). Thus, in various embodiments, a stathmin knockdown approach can be combined with any Stat5A-enhancing nucleic acid strategy or other Stat5A-enhancing strategy hereof.

Other approaches for control of stathmin expression that have been developed can be employed in place of a stathmin knockdown approach hereof. For example, in some embodiments, a stathmin anti-sense or siRNA-based stathmin gene silencing approach can be used. See, e.g., E. Alli et al., Silencing of stathmin induces tumor-suppressor function in breast cancer cell lines harboring mutant p53, Oncogene [Epub ahead of print] (Aug. 14, 2006). Alternatively, a stathmin RNA-degrading activity, such as an anti-stathmin ribozyme, can be expressed from a recombinant construct introduced into a target cell. See, e.g., S. J. Mistry et al., Development of ribozymes that target stathmin, a major regulator of the mitotic spindle, Antisense Nucl. Acid Drug Dev. 11(1):41-9 (February 2001).

### Nucleic Acid Vectors

A vector can be used to deliver the nucleic acid construct(s) to motor neuron cells. In various embodiments, the vector can be a recombinant viral vector, containing either a full or partial complement of viral chromosomal nucleic acid. See, e.g., T. Federici & N. M. Boulis, Gene-based treatment of motor neuron diseases, Muscle & Nerve 33(3):302 (2006). In the case of virulent viruses for use as, or in forming, viral vectors, these can contain a partial complement of viral chromosomal nucleic acid and can be non-virulent, in various embodiments hereof. Among useful viruses for forming recombinant viral vectors are adenoviruses (AV), adeno-associated viruses (AAV), herpes viruses, and lentiviruses; and in recombinant adenoviral, herpes viral, and lentiviral vectors, these can be non-virulent. See, e.g., G. Haase et al., Gene therapy of murine motor neuron disease using adenoviral vectors for neurotrophic factors, Nature Med. 3:429-436 (1997); A. M. Vincent et al., Adeno-associated viral-mediated insulin-like growth factor delivery protects motor neurons in vitro, Neuromolec. Med. 6(2-3):79-85 (2004), and R. J. Mandel et al., Recombinant adeno-associated viral vectors as therapeutic agents to treat neurological disorders, Molec. Ther. 13(3):463-83 (March 2006); D. S. Latchman, Herpes simplex virus vectors for gene therapy in Parkinson's disease and other diseases of the nervous system, J. R. Soc. Med. 92(11):566-570 (November 1999); and L. F. Wong al., Lentivirus-mediated gene transfer to the central nervous system: therapeutic and research applications, Hum. Gene Ther. 17(1):1-9 (January 2006).

In various embodiments, exemplary viruses for use in preparing a viral vector can be: a first or second generation adenovirus or an Epstein-Barr virus or herpes simplex virus. In various embodiments, an AAV or a second generation AV can be used to form a recombinant viral vector hereof. See, e.g., K. N. Barton et al., Second-generation replication-competent oncolytic adenovirus armed with improved suicide genes and ADP gene demonstrates greater efficacy without increased toxicity, Molec. Ther. 13:347-56 (2006); and R. Alba et al., Gutless adenovirus: last-generation adenovirus for gene therapy, Gene Ther. 12 Suppl.(1):S18-27 (October 2005).

In various embodiments, a Stat5A nucleic acid vector hereof can further comprise, or can further be administered with an additional vector comprising, any one or more of a SMN2 gene, an SMN2-splicing nucleic acid, or a stathmin knockdown nucleic acid. Similarly, administration of a Stat5A-enhancing compound can be performed in conjunction with administration of a Stat5A nucleic acid vector hereof, or with a nucleic acid vector containing any one or more of an SMN2 gene, an SMN2-splicing nucleic acid, or a stathmin knockdown nucleic acid.

Such genetic vectors can be administered once or more than once in a course of treatment, and the same vector can be administered each time or a different vector can be used. Such vectors can be administered in conjunction with a further, non-genetic-vector therapeutic agent, whether a pharmaceutical, nutraceutical, or other medically acceptable beneficial substance. In various embodiments, such further agent(s) can be any one or more of substances that directly or indirectly: (1) enhance Stat5A gene transcription, Stat5A gene transcript processing/splicing, Stat5A expression, or Stat5A activity; (2) enhance SMN2 transcription, SMN2 transcript processing (e.g., splicing), SMN expression, or SMN activity; or (3) inhibit stathmin gene transcription, stathmin gene transcript processing/splicing, stathmin expression, or stathmin activity.

### Stat5A Enhancing Compounds

In various embodiments hereof, a Stat5A-enhancing compound can be employed. In various embodiments, a Stat5A-enhancing compound can increase the level of activation of Stat5A; such a compound can be referred to herein as a Stat5A activator. In some embodiments, a Stat5A-enhancing compound can increase the level of Stat5A transcription.

Examples of Stat5A-enhancing compounds useful herein include, as exemplary Stat5A activators: interferon-alpha (IFNα); interleukins IL-2, IL-3, IL-5, IL-6, IL-7, and IL-15; granulocyte/macrophage-colony stimulating factor (GM-CSF); growth hormone (GH); epidermal growth factor (EGF); erythropoietin (EPO); prolactin (PRL); thrombopoietin (TRP); trichostatin A (TSA); aclarubicin; sodium vanadate; and combinations thereof. In various embodiments employing a biomolecule-type Stat5A-enhancing compound, such as a cytokine or peptide hormone, the compound can be selected to be homogenous to the species to be treated. For example, in the case of human subjects, a biomolecule-type Stat5 activator can be chosen from: human interferon-alpha (IFNα); human interleukins IL-2, IL-3, IL-5, IL-6, IL-7, and IL-15; human granulocyte/macrophage-colony stimulating factor (hGM-CSF); human growth hormone (hGH); human epidermal growth factor (hEGF); human erythropoietin (hEPO); human prolactin (hPRL); human thrombopoietin (hTRP); and combinations thereof.

In various embodiments, a Stat5A-enhacing compound can be administered in conjunction with a genetic vector, as described above, or with a further non-genetic-vector therapeutic agent, such as any one or more of those medically acceptable: (1) Stat5A-enhancing compounds, i.e., substances that directly or indirectly enhance Stat5A gene transcription, Stat5A gene transcript processing/splicing, Stat5A expression, or Stat5A activity; (2) SMN2-enhancing compounds, i.e. substances that directly or indirectly enhance SMN2 transcription, SMN2 transcript processing (e.g., splicing), SMN expression, or SMN activity; or (3) stathmin-inhibiting compounds, i.e. substances that directly or indirectly inhibit stathmin gene transcription, stathmin gene transcript processing/splicing, stathmin expression, or stathmin activity. Exemplary SMN2-enhancing compounds include histone deacetylase (HDAC) inhibitors, useful examples of which include: sodium butyrate, valproic acid, sodium phenylbutyrate, suberoylanilide hydroxamic acid, suberic bishydroxamic acid, m-carboxycinnamic acid bishydroxamide, and 4-dimethylamino-N-(6-hydroxycarbamoyl-hexyl)-benzamide.

### NUCLEOBASE Probes

In various embodiments, a nucleobase probe useful in binding assays to screen for, or to competitively inhibit binding by, Stat5A proteins can be provided that comprises a base sequence of a novel CTCNNNTAA motif hereof or the complement thereof, or the RNA base equivalent to either of these. In various embodiments, a nucleobase probe can comprise DNA, RNA, or a nucleic acid analog. A nucleic acid analog can be any known in the art and exemplary types include: locked nucleic acids, peptide nucleic acids (also called polyamide nucleic acids), or other nucleobase-bearing polymers that can provide a nucleic acid-type arrangement of nucleobases pendant to a polymer backbone. Nucleobase probes can in some embodiments hereof be detectably labeled. The detectable label can be any known useful in the art, e.g., an antigen, a fluorophore, or a colored or colorable moiety.

### Methods

The compounds, nucleic acids, and vectors hereof can be used in methods for treating SMA or another SMN-deficiency. In various embodiments, a therapeutically effective amount of a pharmaceutically acceptable Stat5-enhancing substance(s) can be administered to treat SMA by enhancing the transcription/expression of a Stat5 gene, or the activation level of the Stat5 protein, or both. In various embodiments, a therapeutically effective amount of a pharmaceutically acceptable recombinant genetic vector, comprising at least one copy of a host-cell-expressible (e.g., neuron-expressible) gene encoding Statt5A can be administered to treat SMA by increasing the copy number of Stat5A gene(s), and/or to provide upon expression an improved Stat5A protein, such as a constitutively activated Stat5A. Other Stat5A polypeptides, and their coding sequences, can be obtained by mutation and/or recombination, such as can be employed in a directed evolution process, according to any method known therefor in the art.

In some embodiments, a constitutively activated Stat5A can be a Stat5A comprising (1) Phe710 and at least one of Arg298 or Gly150, or (2) His642, according to the numbering of SEQ ID NO:3; or comprising (3) Phe710 and Arg298, according to the numbering of SEQ ID NO:3. In some embodiments, a Stat5A*6 protein can be used as a constitutively activated Stat5A.

In some embodiments of a genetic vector-based therapeutic method hereof, the genetic vector can contain, in addition to the Stat5A enhancing nucleic acid, such as a Stat5A-encoding gene or constitutive Stat5A-encoding gene, a further polynucleotidyl element that is beneficial to SMN-deficiency-afflicted (e.g., SMA-afflicted) subjects. The further element can in some embodiments comprise a Stathmin inhibitor. The Stathmin inhibitor can be a Stathmin expression inhibitor, which can in some embodiments be expressible by the target/host cell. The further element can in some embodiments comprise an Stathmin-specific RNAi nucleic acid, e.g., an shRNA for Stathmin knock-down. In some embodiments, an antisense or RNAi nucleic acid can comprise a Stathmin target sequence of CGTTTGCGAGAGMGGATA (nt728-746 of SEQ ID NO:10) or it complement or the RNA base equivalent of either of these.

In various embodiments, the genetic vector can be a viral vector; and in some embodiments, this can be chosen from the adenoviral, adeno-associated viral, herpes viral, or lentiviral vectors. Second generation adenoviral vectors are exemplary types thereof. In some embodiments an adeno-associated viral vector can be used.

A method according to various embodiments of the present invention can be practiced on any subject in need thereof. For example, a therapeutic method hereof for increasing SMN by targeting Stat5 can be practiced on a human or animal, preferably a mammalian, subject exhibiting SMN deficiency. In various embodiments, the subject can be human. The route of administration can be any known useful for the purpose. For example, a genetic vector can, in some embodiments, be administered parenterally. In embodiments in which the vector is targeted to motor neurons, it can be administered, e.g., by injection to the immediate environment of the neuron, such intramuscularly to a muscle adjacent to the neuron, by infusion to the cerebrospinal fluid, or by injection (e.g., microinjection) to the neuron itself. In some embodiments, peptide hormones, cytokines, or small molecule compounds can be administered orally, enterically, topically, or parenterally.

In various embodiments, a method hereof for identifying a candidate compound for treatment of SMA can be performed by contacting a test substance, e.g., a compound from a library of test compounds, with a mammalian cell that is Stat5(+) and that contains an expressible, Stat5-activatable target nucleic acid whose promoter contains at least one Gamma-Activated Sequence (GAS) element and at least one CTCNNNTAA motif, with contact occurring under conditions in which Stat5 can be activated (e.g., by Tyr phosphorylation in cyto). When an increase in the level of expression (transcription or translation) of, or in the level of expression-dependent phenotype from, the target nucleic acid results, the substance is identified as a candidate. The promoter used can be a SMN promoter, e.g., an SMN2 promoter, and this can be in operative attachment to a native SMN coding sequence or to another coding sequence, such as that of a reporter protein (e.g., luciferase, GFP, and the like).

In some embodiments, the mammalian cell can be a Stat5(+)/SMN2(+) cell. In various embodiments of an assay employing such a cell, the detection can involve assaying the level of SMN2 transcripts, the level of SMN or SMNΔ7 protein, or the occurrence of nuclear gems in the cell nucleus.

The GAS element(s) of the promoter can have a sequence of any GAS element known in the art. For example, GAS element(s) can have a sequence of any one of ttcnnn(n)gaa, ttcnnn(n)gag, or ttcnnn(n)gta, i.e. having a 9- or 10-nucleotide motif as indicated; the 10 nt motifs are SEQ ID NOs:16-18, respectively. In various embodiments, the GAS element(s) can have a sequence of any one of ttcynrgaa, ttcynrgag, or ttcynrgta, and in some embodiments, the ynr segment thereof can have a sequence of any one of ‘cng’ or ‘cna.’ In some embodiments, the GAS element can have a sequence of any one of ttccaggag or ttcctagta. Where more than one GAS element is present in a promoter, these can be independently selected, as can be done for multiple CTCNNNTAA motifs in a given promoter.

In various embodiments, a method hereof for identifying a candidate Stat5-regulated gene (or a promoter thereof can be performed by contacting a cell-derived (e.g., gene-containing) polynucleotide sample, with a nucleobase probe comprising a base sequence of a novel CTCNNNTAA motif hereof or the complement thereof, or the RNA base equivalent to either of these, with contacting being performed under conditions in which the probe can specifically hybridize to a sequence in the sample that is complementary thereto. After washing to remove non-specifically bound probes, detection of hybrids thereby identifies candidate CTCNNNTAA-containing promoters and thus candidate Stat5-regulated genes. These can be further confirmed by, e.g., sequence analysis to identify putative promoter elements, origin of transcription, and the like and comparing the positions of these to that of the probe binding site. Polynucleotide samples can be prepared for hybridization either with or without polynucleotide fragmentation.

In various embodiments, a method hereof for identifying a candidate Stat5 protein, or other candidate transcription factor, can be performed by contacting a CTCNNNTAA motif-containing nucleobase probe hereof with a polypeptide, e.g., a polypeptide having the amino acid sequence of Stat5 or an amino acid sequence at least 70% identical thereto, under conditions in which the polypeptide can specifically bind to the motif sequence to form a complex. After washing to remove non-specifically bound probes, detection of complexes thereby identifies candidate Stat5 (or other transcription factor) proteins.

In various embodiments, a method hereof for identifying a candidate Stat5 protein, or other candidate transcription factor, can be performed by contacting a CTCNNNTAA motif-containing nucleobase probe hereof with a polypeptide, e.g., a polypeptide having the amino acid sequence of Stat5 or an amino acid sequence at least 70% identical thereto, under conditions in which the polypeptide can specifically bind to the motif sequence to form a complex. After washing to remove non-specifically bound probes, detection of complexes thereby identifies candidate Stat5 (or other transcription factor) proteins.

### EXAMPLES

The following examples are non-limiting and serve to illustrate some embodiments of the technology.

### Materials and Methods

Chemicals used to treat SMA-like MEFs or SMN2-NSC34 cells were purchased from Sigma (St. Louis, Mo.) or Calbiochem (San Diego, Calif.). Mouse Stat5A dsRNA was purchased from Dharmacon (siGENOME™, Lafayette, Colo.). Genetic techniques can be performed according to commonly known methods of nucleic acid manipulation, such as those described in: Sambrook & Russell, Molecular Cloning: A Laboratory Manual (2003, Cold Spring Harbor Lab., NY); Ausubel et al. (eds.), Current Protocols in Molecular Biology (2006, Wiley Interscience, NY); and Berger & Kimmel (eds.), Methods in Enzymology 162 (1987, Academic Press, San Diego, Calif.). Pharmaceutical formulations for administration can be prepared by any useful method known in the art, such as those described in: Remington: The Science and Practice of Pharmacy (2005, Lippincott Williams & Wilkins, Philadelphia, Pa.); R. C. Rowe et al., Handbook of Pharmaceutical Excipients (2005, APHA Publications, Washington, D.C.); and Goodman & Gilman's The Pharmacological Basis of Therapeutics (2001, McGraw-Hill Professional, New York, N.Y.). An SMA mouse model that can be used herein can be generated as described in H. Li et al., Nat. Genet. 24(1):66-70 (January 2000), and in U.S. Pat. No. 6,245,963 to Li et al.

Generation of anti-human SMN antibody. The pQE expression system (Qiagen, Valencia, Calif.) was used to express human full length SMN protein in E. coli M15. Induction and purification of SMN protein by affinity chromatography on nitrilotriacetic acid (NTA)-chelating agarose were conducted according to manufacturer's protocols. Purified SMN protein was injected into rabbits with Freund's complete adjuvant (Sigma, St. Louis, Mo.), and antisera obtained were used for Western blot analysis. 20 μg of total protein from cell extracts (3T3 or 293T) was analyzed. Proteins blotted onto polyvinylidene difluoride membranes were incubated with at 1/1,000 dilution and labelled with an HRP-conjugated anti-rabbit secondary antibody (Chemicon, Temecula, Calif.).

Constructs. Human SMN2 gene (35.5 kb) was digested from human SMN2 BAC clone 7C by using BamHI and was inserted into the multiple cloning site of Super COS I expression vector (Stratagene, La Jolla, Calif.). For luciferase assay, the SMN2 promoter (5.4 kb) was digested out using NheI and XhoI and ligated into the pGL3-basic vector (Promega, Madison, Wis.). The pMX-puro-Stat5A1*6 plasmid was a gift from Dr. T. Kitamaura, (University of Tokyo, Tokyo, Japan). The cDNA for the Stat5A1*6 (constitutive activation mutant with H299R/S711F) was sub-cloned into pGEM-T-Easy vector (Promega, Madison, Wis.) using a primer set: Stat5A1*6 forward: 5′-CATGGCGGGCTGGATTCA-3′ (SEQ ID NO:19) and Stat5A1*6 backward: 5′-TCAGGACAGGGAGCTTCT-3′ (SEQ ID NO:20). The restriction enzymes Not I and Spe I were used to excise a 2.3 Kb fragment and were inserted into pFlag-CMV2 expression vector (Sigma, St. Louis, Mo.).

Cell culture and chemical/dsRNA treatment. Mouse embryonic fibroblasts (MEFs) were prepared using the standard protocol. Briefly, E13.5 day embryo was isolated; the uterine deciduas were cut away, and the yolk sac was removed. The embryo was then scraped out to remove non-fibroblastic tissue, and the head severed for genotyping. Embryo body was minced in 0.25% Trypsin-EDTA and incubated for 30 min. It was then added to MEF culture media, and the cell suspension spun for ˜5 min at 1,000 rpm in the tissue culture centrifuge to pellet cells. The supernatant was aspirated off and the cell pellet immediately resuspended in 10 mL of fresh MEF culture media. The MEFs were allowed to reach confluency so the cells could be passaged for further experiments. Cultured MEFs and SMN2-NSC34 cells were maintained in Dulbecco's modified Eagle medium (Invitrogen, Carlsbad, Calif.) containing 10% heat-inactivated fetal bovine serum (Hyclone, Logan, Utah) and 1% penicillin-streptomycin (Invitrogen, Carlsbad, Calif.) and were incubated at 37° C. in a 5% CO₂ humidified atmosphere. The cells were plated the day preceding treatment with each chemical and harvested at the indicated time.

For the Stat5A knockdown experiment, SMN2-NSC34 cells were grown to 70% confluence in a 12 well culture plate and treated with dsRNA for 48 hours, and then treated with sodium vanadate for 4 hours. Later, duplicated cells were harvested for RT-PCR or Western blot analysis. EB-virus transformed Normal and SMA patient lymphocytes were cultured in α-MEM (Invitrogen, Carlsbad, Calif.), 10% heat inactivated fetal bovine serum (Hyclone, Logan, Utah), 1% penicillin-streptomycin as previously described (23). Isolation and primary culture of motor neuron cells and genotyping of individual embryos were also carried out as described in: R. I. Schnaar & A. E. Schaffner, Separation of cell types from embryonic chicken and rat spinal cord: characterization of motoneuron-enriched fractions, J. Neurosci. 1 (2):204-17 (February 1981); Y. Arakawa et al., Survival effect of ciliary neurotrophic factor (CNTF) on chick embryonic motoneurons in culture: comparison with other neurotrophic factors and cytokines, J. Neurosci. 10(11):3507-15 (November 1990); and S. Wiese et al., The role of p75NTR in modulating neurotrophin survival effects in developing motoneurons, Eur. J. Neurosci. 11 (5):1668-676 (May 1999).

Briefly, the ventrolateral parts of individual lumbar spinal cords were dissected and transferred to HBSS (Hank's Balanced Salt Solution, Sigma, St. Louis, Mo.). After treatment with trypsin (0.05%, 15 min) (Invitrogen, Carlsbad, Calif.) single-cell suspensions were triturated and the cell suspension passed through a nylon mesh (100 μm pore size). The cells were overlaid on 10% Histopenz (Sigma, St. Louis, Mo.) in HBSS. The Histopenz cushion was centrifuged for 20 min at 250 g, and cells from the inter-phase were taken out and transferred to culture medium. Cells were plated at a density of 2000 cells/cm² in a 4-well chamber slide (Nalge Nunc), pre-coated with poly-ornithine and laminin (Sigma, St. Louis, Mo.). Cells were grown in neurobasal medium (Invitrogen, Carlsbad, Calif.) with 5% horse serum (Invitrogen, Carlsbad, Calif.), 5% fetal bovine serum and 500 μM glutamax (Invitrogen, Carlsbad, Calif.) and 1% penicillin-streptomycin at 37° C. in a 5% CO₂ atmosphere. Fifty percent of the medium was replaced at day 1 and changed every second day. Cells were cultured in the presence of ciliary neurotropic factor (CNTF) and brain-derived neurotropic factor (BDNF) (10 ng/mL each) (CytoLab Ltd, Rehovot, Israel). Motor neuron cells were cultured for three days and harvested for further transfection or immunocytochemical analysis.

Semi-quantitative PCR/RT-PCR. The genomic DNA from E13.5 SMA embryos was extracted. A specific primer pair, SMN forward 5′-TGTAGTGGAAAGTTGGGGAC-3′ (SEQ ID NO:21) and SMN backward 5′-CCTGGCATTGGGGGTGGTGGAGG-3′ (SEQ ID NO:22), was designed for recognition of both murine Smn and human SMN2. The PCR program initially started with a 95° C. denaturation for 10 min, followed by 19 to 26 cycles of 95° C./1 min. 53° C./1.5 min, 72° C./2 min to assay the linear range for both Smn and SMN2. For RT-PCR assay, total RNA was extracted at indicated time points from SMN2-NSC34 cells with sodium vanadate, TSA, and aclarubicin treatment or with an increasing amount of Stat5A1*6 transfection by using the TRizol reagent (Invitrogen, Carlsbad, Calif.). To amplify the exon7 inclusion/exclusion form of SMN2 transcripts, RT-PCR were performed using a primer set P5P6 as previously described (15). The transcript from the mouse glyceraldehyde-3-phosphate dehydrogenase (Gapdh) gene or the β-Actin gene was amplified using the primer pairs: Gapdh forward: 5′-CCCTTCATTGACCTCAACTA-3′ (SEQ ID NO:23), and backward: 5′-CCAAAGTTGTCATGGATGAC-3′ (SEQ ID NO:24) (56° C.), and β-Actin forward: 5′-ATGGTGGGMTGGGTCAGMGGAC-3′ (SEQ ID NO:25), and backward 5′-CTCTTTGATGTCACGCACGATTTC-3′ (SEQ ID NO:26) (59° C.), and this allowed control of equal amounts of template. To analyze total SMN2 transcripts, primer sets were designed to specifically recognize SMN2 exon2a and exon6: Exon2a forward: 5′-CTGACATTTGGGATGATACAGCAC-3′ (SEQ ID NO:27) and Exon6 backward: 5′-TGGTGGAGGGAGAAAAGAGTTCC-3′ (SEQ ID NO:28). The PCR program initially started with a 95° C. denaturation for 5 min, followed by 15 to 25 cycles of 95° C./1 min, 54° C./1 min, and 72° C./1.5 min to assay the linear range for SMN2. The resulting PCR products were electrophoresed on 1.2% or 1.5% agarose gels in TBE buffer [89 mM Tris-base pH 7.6, 89 mM boric acid, 2 mM EDTA] and stained with ethidium bromide [10 μg/mL] and photographed on top of a 280 nm UV light box. The gel images were digitally captured with a CCD camera and analyzed with the Alphalmager™. To specify the SMN2 copy number, the SMN2 signal was normalized with the endogenous mouse Smn signal. RT-PCR values are presented as a ratio of the FL-SMN2 signal divided by Δ7-SMN2 in the selected linear amplification cycle normalized by Gapdh, or β-Actin signal. Relative total SMN2 transcript levels were determined from Stat5A1*6 transfected SMN2-NSC34 cells in a minimum of three independent experiments. Differences in ratios were determined to be significant by an independent two-tailed t-test, with *, P<0.05, **, P<0.005, ***, p<0.001.

Western blot analysis. Cells treated by dsRNA, compounds or Stat5A1*6 transfected were detached by scraping, pelletting and rinsing in PBS. Cell pellets were collected after centrifugation and lysed on ice in modified RIPA buffer (50 mM Tris-HCl (pH7.4), 1% NP-40, 0.25% deoxycholic acid, 0.15M NaCl, 1 mM EDTA, 1 mM PMSF/NaF/sodium orthovanadate, and protease inhibitors cocktail (Roche, Mannheim, Germany) for 30 min. After centrifugation, the supernatants were collected and kept frozen at −20° C. Protein concentrations were determined by Bio-Rad protein assay method. For Western blot analysis, protein samples were boiled for 5 min and electrophoresed on 8% or 10% SDS-polyacrylamide gel in a 1× running buffer (25 mM Tris, 192 mM glycine, 3.4 mM SDS, pH 8.3) and subsequently electrotransferred to Polyvinylidene Fluoride tansfer membrane (Pall, Pensacola, Fla.) using a TE 22 mini-tank transfer unit (Amersham Biosciences, San Francisco, Calif.) at 35 volts overnight in transfer buffer (25 mM Tris, 192 mM glycine, 20% methanol, pH8.3). The blotting membranes were incubated in blocking solution (PBS, 5% non-fat milk, 0.2% Tween-20) for 1 hour at room temperature, and then incubated in the same solution with the primary antibody (human-specific SMN/1:1000; SMN/1:5000, Transduction Laboratories, Lexington, Ky.; phospho-Stat5a/b/1:1000, Cell signaling, Beverly, Mass.; phospho-Jak2/1:1000, Upstate, Lake Placid, N.Y.; Flag BioM2/1:1000, Sigma, St. Louis, MO; c-myc/1:200, Santa Cruz Biotech, Santa Cruz, Calif.; α-Tubilin/1:10000, Upstate, Lake Placid, N.Y., MO), overnight at 4° C. The membranes were washed and incubated in the blocking solution with the proper HRP-conjugated secondary antibody at 1/5000 dilution (Chemicon, Temecula, Calif.) for 1 hour at room temperature. After washing three times in PBS containing 0.1% Tween-20, the signals were visualized by autoradiography (Fuji Medical X-ray film, Fuji Photos, Tokyo) using enhanced chemi-luminescence (ECL detection system; Perkin-Elmer, Boston, Calif.). Western blot quantification was performed by scanning the auto-radiographs with a computerized densitometer. Signal intensities were determined by densitometry analysis (Fuji film LAS-1000 plus pictography) using the program Phoreticx 1D (Phoreticx International).

Reporter analysis. For SMN2 gene promoter derived luciferase assay, the pSMN2-luciferase vector (0.75 μg) was co-transfected with pSV40-Renilla luciferase vector (0.25 μg) and flag-tagged Stat5A1*6 (2 ug) into the 2×10⁵ NSC34 cells using lipofectAMINE2000 reagent (Invitrogen, Carlsbad, Calif.). Cells were harvested 24 hours after transfection and relative luciferase activities were measured according to the manufacturer's standard procedures (Promega, Madison, Wis.). Statistical analysis comparing SMN2 promoter activity between Stat5A1*6-transfected to non-transfected NSC34 cells was conducted using an independent two-sided t-test, with ***, P<0.0001.

In vitro binding assay. The binding assay was performed by using the (EMSA alternative) NoShift Transcription factor assay kit (Novagen). Briefly, two oligonucleotides that define a putative Stat5 binding site in the SMN2 promoter were synthesized and the 3′-end labeled with biotin. After annealing, the dsDNA was incubated with the chemical treated or non-treated SMN2-NSC34 nuclear extract for 30 minutes on ice, and then transferred to a streptavidin plate and incubated for 1 hour at 37° C. 1 hour later, the primary antibody (anti-phospho-Stat5/1:200, Santa Cruz, Calif.) was added and incubated for 1 hour at 37° C. After washing, the secondary antibody conjugated with horseradish peroxidase was added and incubated for 30 minutes at 37° C. After washing 5 times, TMB substrate was added and incubated at room temperature in the dark until the blue color developed and then the reaction stopped by adding 1 N HCl; finally, the absorbance at 450 nm was measured with PowerWave 340 reader (BIO-TEK, instruments). Each experiment was performed three times and SEM was calculated. Differences in ratios were determined to be significant by an independent two-tailed t-test, with **, P<0.01 and ***P<0.0001.

Transfection and Immunocytochemical analysis. The Super COS I-SMN2 was first transfected into NSC34 cells using the LipofectAMINE™2000 following the manufacturer's protocol and several transfectants were obtained through 500 μg/ml G418 (Calbiochem, San Diego, Calif.) selection. Transfectant D9 was used for further studies. For SMA patient lymphocyte transfection, cells were pelleted (˜5×10⁶) cells by centrifugation at 1000 rpm for 3 min and washed twice with ice-cold PBS. The cells were then re-suspended in the pellet in 600 μL PBS, and 10 μg of DNA was added (empty vector or flag tagged Stat5 A1*6 constructs). The cell suspension was then transferred to a cold 0.4 cm gene pulser cuvette (Bio-Rad, Hercules, Calif.) and the cells were electroporated at 0.95 kV/27 μF. The electroporated cells were then cultured for 36 hours and fixed with 4% PFA for 10 minutes and permeablized on 0.3% Triton-X 100 in PBS for 5 mins. After blocking with 3% BSA, the cells were incubated overnight at 4° C. with the following primary antibodies: Flag polyclonal (1:500; Sigma, St. Louis, Mo.) and SMN (1:500, Transduction laboratories, Lexington, Ky.). Cells were then washed three times with TBS-T (20 mM Tris-HCl, pH 7.4, 137 mM NaCl, and 0.1% Tween 20) and incubated for 1 hour at RT with appropriate fluorescence dye conjugated secondary antibodies (1:500; Molecular probes, Eugene, Oreg.). DAPI (Sigma, St. Louis, Mo.) was used for nucleus staining. After mounting with fluorescent mounting medium (DAKO, Carpinteria, Calif.), the suspended cells were transferred into a chamber, and images were obtained with a LSM 510 laser-scanning confocal microscope. The LSM5 Image Browser software was used for image acquisition. Statistical analysis comparing gem number of control vector transfected group to Stat5A1*6 transfected lymphocytes was conducted using an independent two-sided t-test, with *, P<0.01. For primary motor neuron transfection, 0.1M polyethylenimine reagent (Sigma, St. Louis, Mo.) was used as previously reported (64). Opti-MEM (Invitrogen, Carlsbad, Calif.) diluted plasmid DNA (Stat5A1*6) was added to opti-MEM diluted polyethylenimine solution (PEI, in 5% glucose) in the same volume while vortexing (giving rise to an N/P ratio of 10). After 15 min incubation, the mixture was added into the culture medium. After 48 hours, cells were harvested for immunocytochemical analysis. Neurite outgrowth was quantified by using Neuron, see J E. Meijering et al., Cytometry A. 58, 167-176 (2004), a JAVA program for neurite tracing and quantification (http://imagescience.bigr.nl/meijering/software/neuronj/) described in J. M. Harper et al, Proc. Nat'l Acad. Sci. USA. 101, 7123-7128 (2004).

Neurons and their axons were identified by using the HB9, βIII-Tubulin, ChAT, or Neurofilament-H antibodies (Chemicon, Temecula, Calif.). Axon length was measured by tracing and recording the length of all βIII-Tubulin and ChAT positive axons. Cells with axons that were not in full view were not included. Total axon length was then divided by the total number of cells, generating a mean axon length per cell within each test group. The SEM was determined and mean axon outgrowth was plotted as a percentage of the control group. Statistical significance: ***, P<0.0001, when Stat5A1*6 transfected SMA motor neurons were compared with control vector transfected groups.

### Example 1—Screening of Compounds for SMN2-Enhancing Activity

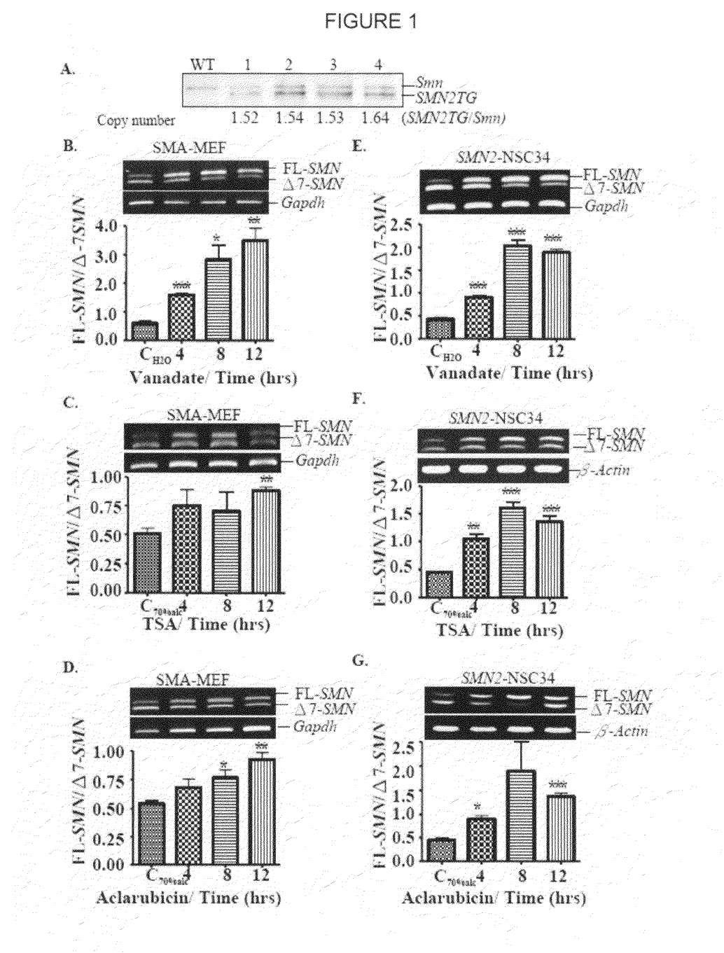

Screening of compounds for SMN2-enhancing activity was performed as follows. We used SMA-like mouse embryonic fibroblasts (Smn−/−, SMN2) (MEFs) with a similar SMN2 copy number (FIG. 1A, SMN2 copy number=1.54) to mimic Type I SMA patients for the first round screening and SMN2 (35.5 Kb)-transfected motor neuron-like NSC34 cells (SMN2-NSC34) for the second round screening. Following a series of time-course RT-PCR analyses using Gapdh or β-Actin transcript level as internal control, sodium vanadate, TSA, and aclarubicin were all found to influence SMN2 expression in SMA-like MEFs (Table 2, FIG. 1B-D).

TABLE 2
Ten Known Compounds Tested in SMA-like MEFs and SMN2-NSC34 Cells

| | | | SMN2 | |
|---|---|---|---|---|
| | | | splicing pattern | |
| Chemical | Solvent | Dosage | MEF | SMN2-NSC34 |
| N-Acetyl-L-cysteine | H₂O | | − | N.D. |
| Phorbol-12-myristate-13-acetate | DMSO | | − | N.D. |
| Lectin | D-PBS | | − | N.D. |
| 5′-Aza-2′-deoxycytidine | 50% acetic acid | | − | N.D. |
| Trichostatin A (TSA) | 70% ethanol | 10 nM | + | ++ |
| Sodium butyrate | H₂O | 25 mM | + | + |
| Aclarubicin | 70% ethanol | 80 nM | + | ++ |
| All-trans retinoic acid | 95% ethanol | | − | N.D. |
| Hydroxyurea | H₂O | | − | N.D. |
| Sodium vanadate | H₂O | 50 μM, 100 μM | +++ | +++ |

N.D: not detected;
−: no effect;
+: effective;
+++: most effective.

These three compounds were further tested in SMN2-NSC34 cells and the results were similar to those found in treating SMA-like MEFs (Table 2, FIG. 1E-G). The full length SMN2 expression level was enhanced after treatment with sodium vanadate for 2-4 hours (FIG. 1B, 1E, and data not shown) and the other two compounds for 4-6 hours (FIGS. 1B and 1C). Although the three compounds were all effective, sodium vanadate appeared most efficient because the time taken for the full length SMN2 transcript to increase was the fastest and the relative increase of full length SMN2 transcripts was greater than with TSA or aclarubicin. These results suggested that these three compounds may activate similar mechanisms involved in SMN2 expression in both fibroblasts and neuronal cells.

### Example 2—Screening of Compounds for Signal Molecule Activity

TSA, aclarubicin, and sodium vanadate were further tested for activity toward signaling molecules involved in tyrosine phosphorylation, including protein tyrosine phosphatase (PTP) activity, and in the receptor tyrosine kinase (RTK) cascade, and toward downstream transcription factors in SMN2-NSC34 cells (see Table 3).

TABLE 3
Signaling Pathways Screened in SMN2-NSC34 Cells

| | | Sodium | | | | | |
|---|---|---|---|---|---|---|---|
| Cellular Response | | vanadate | | TSA | | Aclarubicin | |
| Pathway | Factors Tested | 4 hrs | 8 hrs | 4 hrs | 8 hrs | 4 hrs | 8 hrs |
| Protein tyrosine | KAP | +/− | − | N.D. | | N.D. | |
| phosphorylation | PTP1C/SHP1 | +/− | − | | | | |
| | PTP1B | +/− | − | | | | |
| | RPTPβ | +/− | +/− | | | | |
| | RPTPα | +/− | − | | | | |
| | LAR | +/− | +/− | | | | |
| | MKP2 | − | − | | | | |
| Receptor tyrosine | Crk | +/− | +/− | N.D. | | N.D. | |
| kinase (RTK)- | GRB2 | +/− | +/− | | | | |
| mediated signal | GRB14 | +/− | +/− | | | | |
| transduction | NCK | +/− | +/− | | | | |
| | PI3-Kinase | +/− | +/− | | | | |
| | SHC | +/− | +/− | | | | |
| | ShcC | +/− | +/− | | | | |
| MAPK (Mitogen- | ERK1/2 (pT202/pY204) | + | ++ | + | + | +/− | +/− |
| activated protein | JNK (pT183/pY185) | +/− | + | +/− | + | | |
| kinase) signaling | P38 (pT180/pY182) | +/− | + | +/− | + | +/− | +/− |
| Stat family | Stat1 (pY701) | − | +/− | − | − | +/− | +/− |
| | Stat2 (pY705) | +/− | +/− | +/− | +/− | + | +/− |
| | Stat3 (pY705) | + | ++ | + | ++ | + | ++ |
| | Stat5 (pT694) | ++ | +++ | ++ | +++ | ++ | +++ |
| | Stat6 (pT641) | − | − | +/− | − | +/− | − |
| | Jak1 (pY1022/pY1023) | +/− | + | N.D. | | N.D. | |
| | Jak2 (pY1007/pY1008) | + | ++ | + | +/− | + | +/− |

N.D., not determined;
−, decreasing;
+/−, no activation;
+, slight activation;
++, minor activation;
+++, significant activation.

Time course analysis showed that, after treatment with sodium vanadate for 2-4 hours, or TSA or aclarubicin for 4 hours, the phosphorylation level of Stat5 was increased about 6-fold (Na Vanadate) or about 2-fold (others) compared to control cells, and remained activated at 8 hours α-tubulin internal control; data not shown). Therefore, TSA, aclarubicin and sodium vanadate all induce the activation of Stat5A in motor neuron-like NSC34 cells. Also, Jak2, an upstream protein kinase of Stat5, was found activated at 2-4 hours after treatment; thus, it is likely that the Jak2/Stat5 signaling pathway is activated thereby in neuronal cells.

### Example 3—Testing the Effect of Stat5A Activation on SMN2 Expression

A construct encoding a constitutively activated Stat5A mutant (Stat5A1*6) was prepared and different amounts thereof (2-6 μg) were transiently transfected into SMN2-NSC34 cells. The results of semi-quantitative RT-PCR using SMN2 exon2a and exon6 primers, with Gapdh as internal control, showed that both full-length and exon7-deleted SMN2 (SMNΔ7) transcripts increased significantly in a dose-dependent manner (data not shown), and the SMN2 splicing pattern was found to be unchanged. For confirmation, a 5.4 kb SMN2 promoter-derived luciferase expression vector was prepared and co-transfected with Stat5A1*6 into NSC34 cells; pSV40-renilla luciferase and pCMV-Flag vectors were also included to estimate the background activity of the plasmid. The results showed that luciferase activity increased about 3-fold, versus non-transfected control, when Stat5A1*6 was expressed (data not shown).

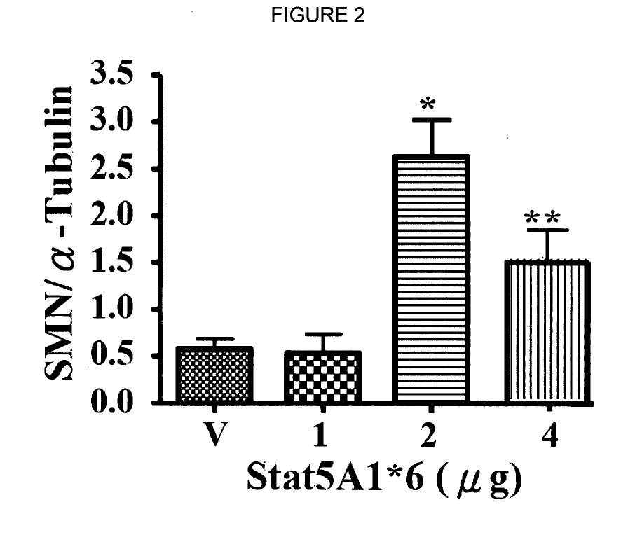

To test whether SMN protein levels also increased during transiently transfected Stat5A1*6 expression in NSC34 cells, we generated a human-SMN-specific antibody having greater specificity toward human as versus murine SMN, as compared with a common, commercial SMN antibody. Immuno-blot analysis therewith of cells receiving increasing amounts (1-4 μg) of Stat5A1*6 showed that SMN protein level, compared to α-tubulin internal control, significantly increased 2.6 and 4.6-fold (FIG. 2) when Stat5A1*6 was expressed.

### Example 4—Screening the SMN2 Promoter for Stat5 Binding Sequences

The promoter sequences of both murine and human SMN genes were analyzed for Stat5 binding sites using MacVector software (Accelrys Inc.). This identified two conserved Stat5 binding sites (TTCNNNGAA and TTCNNNTAA) in the murine promoter (NCBI accession number AF027668), but surprisingly none in the human promoter (NCBI accession number AF027688). Instead, novel elements, having unexpected consensus sequence of CTCNNNTAA, were found in the human SMN2 promoter at nts942-950, nts2474-2482, and nts4403-4411 of SEQ ID NO:8.

We then tested whether or not the Stat5 protein can bind to such novel putative sites. 5 μL samples of nuclear extracts of sodium vanadate-induced SMN2-NSC34 cells were incubated with increasing amounts of a 3′-biotinylated dsDNA probe having the sequence, CCCAGTCTCTACTAAATACAA (SEQ ID NO:12, binding site underlined), resulting DNA-protein complexes were identified using a phospho-Stat5 antibody. Compared to controls, the signal-to-background ratio increased from 2.13:1 to 3.34:1; and the binding assay showed a linear, protein concentration dependence (data not shown).

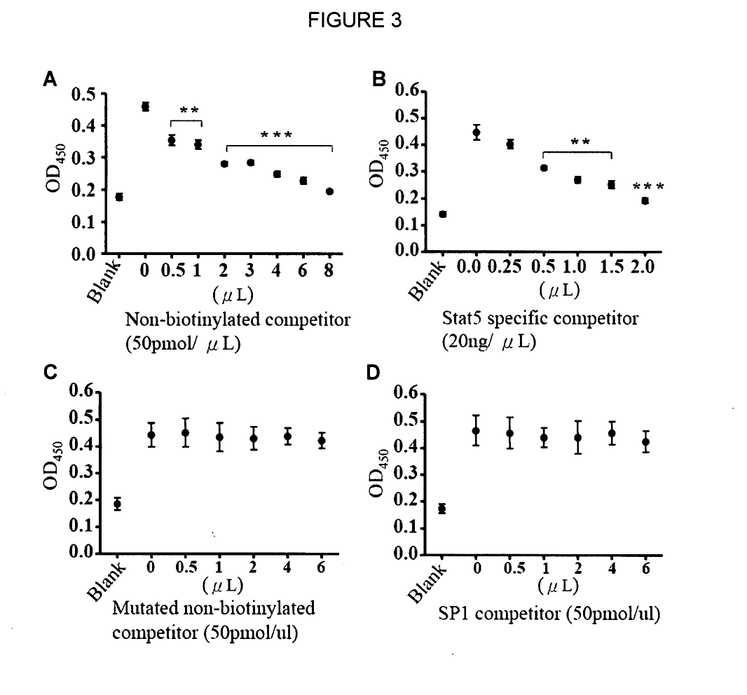

Binding competition analyses were then performed under the same conditions using increasing amounts of: (1) a non-biotinylated novel-Stat5-specific dsDNA probe (SEQ ID NO:12; FIG. 3A); (2) a specific Stat5 consensus binding site dsDNA probe of AGATTTCTAGGAATTCAATCC (SEQ ID NO:13; FIG. 3B); (3) a non-specific transcription factor SP1 consensus binding site dsDNA probe of GCTCGCCCCGCCCCGATCGAAT (SEQ ID NO:14; FIG. 3C); and (4) a mutated, novel-Stat5 dsDNA probe of CCCAGTCTTTACTTAATACAA (SEQ ID NO:15; FIG. 3D); binding sites are underlined. It was found that the Stat5-specific (1) and the Stat5-consensus (2) probes effectively competed for Stat5, but that neither of the SP1 (3) nor mutated Stat5 (4) probes had any effect on binding. Consequently, transcription activator Stat5 does have such novel, specific binding sites within the SMN2 promoter.

### Example 5—Further Characterization of Stat5 Function in Cyto

To further characterize the role of Stat5 in SMN2 regulation, Stat5A dsRNA pre-treatment (48 h) was used to perform RNAi knock-down of endogenous Stat5 expression in SMN2-NSC34 cells. Upon 4 h of 100 μM sodium vanadate treatment of pre-treated cells, SMN level was found decreased 1.72 fold. Full-length SMN2 transcripts remained elevated, yet at a lower level. Stat5A and SMN protein levels, in duplicate samples, were assayed by Western blot and normalized to α-Tubulin; and SMN2 splicing pattern or Stat5A expression was detected by RT-PCR using Gapdh as internal control; the mean of triplicate experiments was calculated (data not shown). These results unexpectedly implicate that sodium vanadate treatment can induce dual pathways: transcriptional activation and alternative splicing.

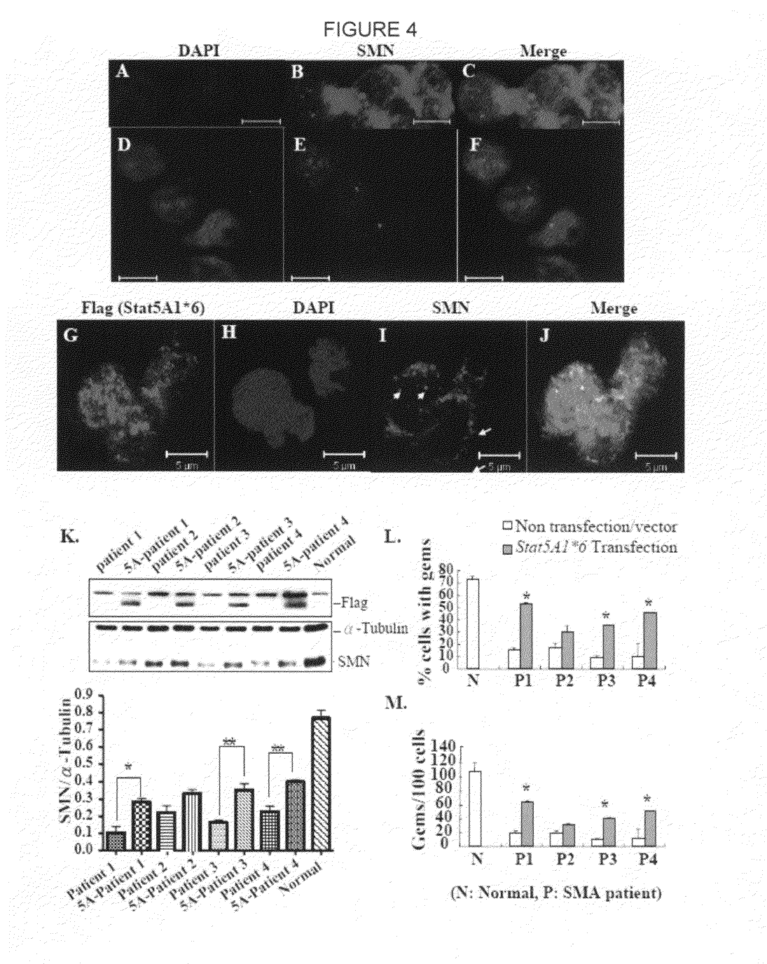

As shown in FIG. 4, expression of the constitutively active Stat5A1*6 in SMN1-deficient SMA patient lymphocytes, which exhibited very low levels of nuclear gems, resulted in a significant increase in the occurrence of nuclear gems as measured by immunocytochemical analysis. Thus, Stat5 activation was found to recover nuclear gems in SMN-deficient cells in vitro. More specifically, FIG. 4 shows that constitutive activation of Stat5A increases the gem numbers in SMA patient lymphocytes. Immunocytochemical analysis of the SMN expression in an EB-virus transformed normal person (4A-C) or type I SMA patient lymphocytes (4D-F). SMN was stained with SMN antibody (4B, 4E) (green). DAPI was used for nuclei staining (4A, 4D). Note that SMN was almost undetectable in type I SMA patient lymphocytes but revealed clear gem nuclear structure and cytosolic signal in normal lymphocytes. Flag-tagged Stat5A1*6 transfected type I SMA patient lymphocytes profoundly increased SMN expression as shown in I (indicated with arrows). Stat5A1*6 was stained with anti-Flag antibody (4G) (red) (Bar: A-J, 5 μm). (4K) Cell lysates from normal person (Normal), four type I SMA patients with (5A-P1 to 5A-P4) or without (P1-P4) Stat5A1*6 transfection were used for Western blot analysis by SMN antibody. Bottom, quantitative analysis of the results from K, three in four SMA patients showed significantly increased SMN expression (patient 1, 3, and 4), but not a profound increase in patient 2. At least 3 experiments are performed and the results represent of mean±SEM. *, P=0.0133 and **, P<0.009 compared with control cells by t-test. The percentage of nuclei with gems (4L) and number of gems per 100 nuclei (4M) in Stat5A1*6 transfected and non-transfected cell lines was evaluated by immunocytochemical analysis. Mean values are shown as determined from at least three experiments for each cell line. The error bars indicate SD. *, P<0.01 when compared with lymphocytes transfected with control vector by t-test.

Defects in axon outgrowth were identified in (Smn+/−, SMN2) and (Smn−/−, SMN2) motor neurons, but not in normal motor neurons, isolated from spinal cords of embryonic day 13.5 mouse embryos, as characterized by ChAT or Hb9 (red) staining, and β-III Tubulin (green) staining of axon processes (data not shown).

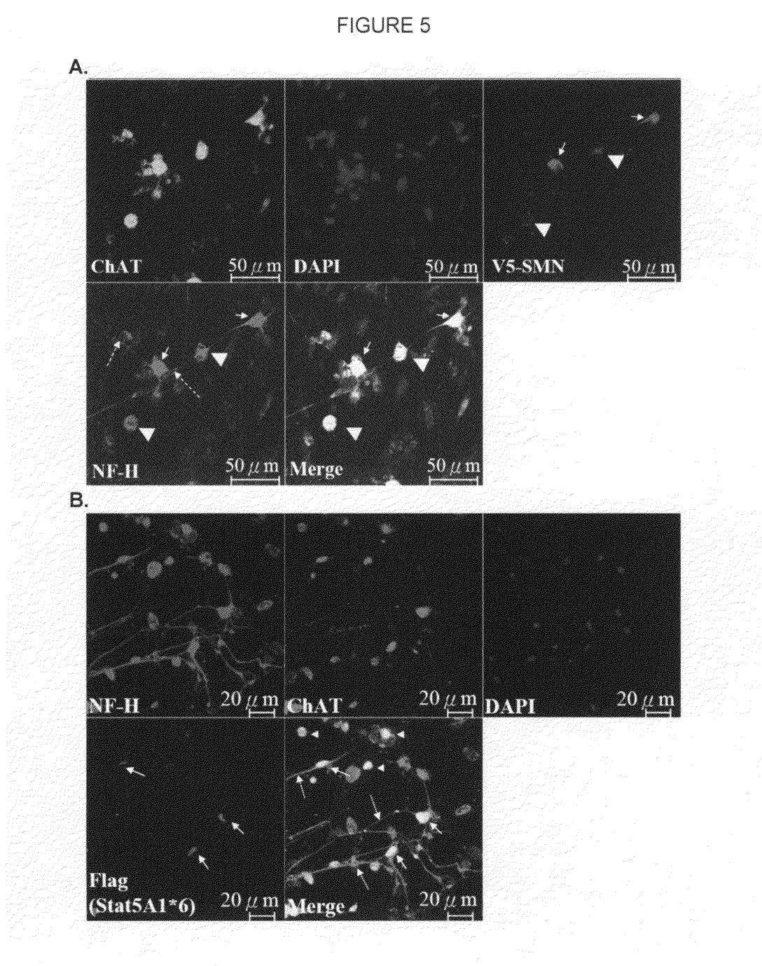

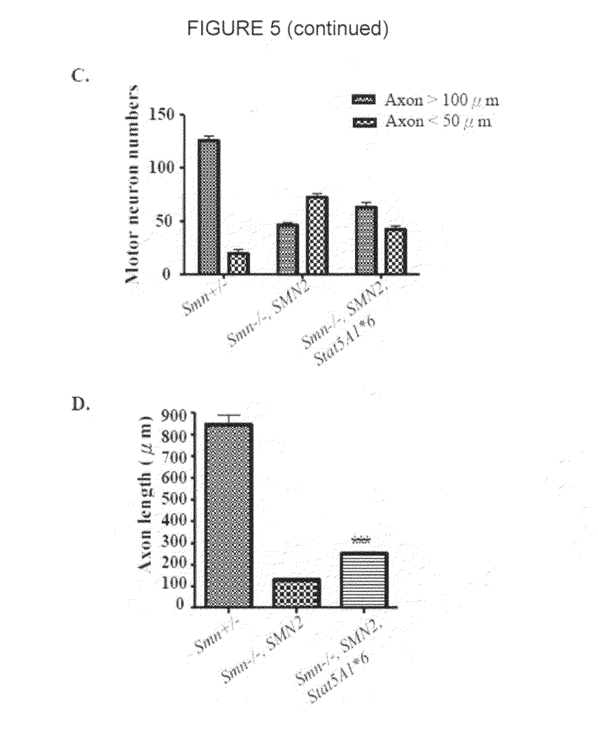

Referring to FIG. 5, a normal neurite phenotype of axon outgrowth was found in normal (Smn+/+) and heterozygous (Smn+/+; SMN2) cultured mouse motor neuron cells, with most (86.9%) showing extended axons (5D; cf. 5C for 13.1% exhibiting a shorter phenotype). Yet, in SMA motor neurons (Smn−/−; SMN2) under the same culture conditions, 38.9% of cells had shorter axons (5D), with the remainder exhibiting axon-less phenotypes (<50 μm), i.e., indistinguishable from dendrites (5C); and no neurite networks were observed. This indicates that SMN expression level correlates closely with motor neuron axon outgrowth phenotypes.

Cultured mouse SMA motor neuron cells were then transiently transfected with V5-tagged SMN expression vector. Immunocytochemistry analysis clearly detected axon outgrowth in only the SMN-transfected SMA motor neurons (FIG. 5A, arrows). The axon length of SMN-recovered SMA motor neurons can extend long distances (FIG. 5A, dotted line) in the same way as Smn+/+ motor neurons. Thus, SMN is a key factor for motor neuron axon extension.

Cultured mouse SMA motor neuron cells were also transfected with a Stat5A1*6-overexpressing construct. Transfected neurons exhibited diminished levels of axon outgrowth defects (FIG. 5B, arrows), with 59.6% of transfected SMA motor neurons exhibiting extended axons (5C). Axon length was also found to be significantly longer compared with SMA motor neurons lacking Stat5A1*6 over-expression (FIG. 5D). Thus, increasing Stat5A expression in motor neurons has been found to diminish axon outgrowth defects and enhance the axon outgrowth phenotype in SMA motor neurons.

More specifically, FIG. 5 demonstrates that constitutive expression of Stat5A enhances neurite outgrowth in SMA motor neurons. (5A) Homozygous mutant motor neurons (Smn−/−, SMN2) with SMN over-expression rescued defects in axon outgrowth, the motor axon extended into long processes (indicated with arrows and dotted lines). Arrowhead indicates mutant SMA motor neurons without SMN transfection. Bar, 50 μm. (5B) Stat5A1*6 transfected SMA motor neurons (Smn−/−, SMN2, Stat5A1*6) also showed remarkable axon extension (indicated with arrows and dotted lines) compared with the Stat5A1*6 non-transfected SMA motor neurons (indicated with arrowheads) and formed a similar axon outgrowth pattern to Smn heterozygous motor neurons (Smn+/−, SMN2). Bar, 20 μm. Motor neurons were characterized with ChAT activity; the axon process was stained with Neurofilament-H. Stat5A1*6 and SMN were stained with Flag tag and V5 tag, respectively. Most heterozygous motor neurons (86.9%, 5C) extended axon (axon length >100 μm) for a long distance (845.2±229 μm, 5D). In SMA motor neurons, only 38.9% cells extended axons and the length was profoundly shorter (148.2±65.07 μm, 5D) than heterozygous motor neurons. However, Stat5A1*6 transfection diminished axon outgrowth defects in SMA motor neurons. 59.6% of Stat5A1*6 transfected SMA motor neurons extended long axons (251.2±97.40 μm, 5D) as compared to other SMA motor neurons. Mean value in each group is shown as determined from total counted motor neuron axons (n=3). ***, P<0.0001 when Stat5A1*6-transfected compared to non-transfected SMA motor neurons, by t-test.

The embodiments and the examples described herein are exemplary and not intended to be limiting in describing the full scope of compositions and methods of the present technology. Equivalent changes, modifications and variations of some embodiments, materials, compositions and methods can be made within the scope of the present technology, with substantially similar results.

What is claimed is:

1. A method for treating spinal muscular atrophy (SMA) or other SMN-deficiency in a subject, the method comprising administering to said subject a therapeutically effective amount of a pharmaceutically acceptable activator of Stat5.

2. The method according to claim 1, wherein the Stat5 activator is chosen from: interferon-alpha (IFNα); interleukins IL-2, IL-3, IL-5, IL-6, IL-7, and IL-15; granulocyte/macrophage-colony stimulating factor (GM-CSF); growth hormone (GH); epidermal growth factor (EGF); erythropoietin (EPO); prolactin (PRL); thrombopoietin (TRP); trichostatin A (TSA); aclarubicin; sodium vanadate; and combinations thereof.

3. A method for treating SMA or other SMN-deficiency in a subject, the method comprising administering to said subject a recombinant genetic vector comprising at least one copy of a host-expressible gene encoding Stat5A.

4. The method according to claim 3, wherein the Stat5A is a constitutively activated Stat5A.

5. The method according to claim 4, wherein the constitutively activated Stat5A is a Stat5A comprising (1) Phe710 and at least one of Arg298 or Gly150, or (2) His642, according to the numbering of SEQ ID NO:3.

6. The method according to claim 4, wherein the constitutively activated Stat5A is a Stat5A comprising Phe710 and Arg298, according to the numbering of SEQ ID NO:3.

7. The method according to claim 4, wherein the Stat5 is Stat5A1*6.

8. The method according to claim 3, wherein the vector is a viral vector

9. The method according to claim 8 wherein the viral vector is an adenoviral, adeno-associated viral, herpes viral, or lentiviral vector.

10. A method for identifying a candidate compound for treatment of SMA comprising
(A) providing (1) a mammalian cell that is Stat5(+) and that contains an expressible, Stat5-activatable target nucleic acid whose promoter contains at least one Gamma-Activated Sequence (GAS) element and at least one CTCNNNTAA motif, and (2) at least one test compound;
(B) contacting the cell with the test compound (1) under conditions in which the test compound can activate Stat5 or (2) under conditions in which the test compound can increase expression of Stat5 and conditions in which Stat5 can be activated; and
(C) detecting the level of expression of the target nucleic acid or of a phenotypic effect resulting from expression thereof,
whereby detection of an increased level of expression of the target nucleic acid or an increase in the phenotypic effect identifies the test compound as a candidate compound for SMA treatment.

11. The method according to claim 10, wherein the mammalian cell is a Stat5(+)/SMN2(+) cell and the detection involves assaying the level of SMN2 transcripts, the level of SMN or SMNΔ7 protein, or the occurrence of nuclear gems in the cell nucleus.

12. The method according to claim 10, wherein the GAS element has a sequence of any one of ttcnnn(n)gaa, ttcnnn(n)gag, or ttcnnn(n)gta.

13. The method according to claim 12, wherein the GAS element has a sequence of any one of ttcynrgaa, ttcynrgag, or ttcynrgta.

14. The method according to claim 13, wherein the ynr segment of the GAS element has a sequence of any one of cng or cna.

15. The method according to claim 14, wherein the GAS element has a sequence of any one of ttccaggag or ttcctagta.

16. A nucleobase probe containing a base sequence of CTCNNNTAA or the complement thereof, or the RNA base equivalent to either of these.

17. The nucleobase probe according to claim 16, wherein the probe comprises DNA or a nucleic acid analog.

18. A method for screening to identify a candidate Stat5-regulated gene, the method comprising:
(A) providing a nucleobase probe containing a base sequence of CTCNNNTAA or the complement thereof, or the RNA base equivalent to either of these.
(B) contacting a gene-containing cell, cell fragment, or polynucleotide preparation with the probe under conditions in which the probe can hybridize specifically to a sequence complementary to the base sequence of (A) to form hybrids, and removing non-specifically hybridized probes therefrom to leave remaining hybrids, and
(C) detecting remaining hybrids and determining that the target sequence to which the base sequence of (A) has bound is located in a gene promoter region,
whereby detection of a remaining hybrid identifies the gene member thereof, or the gene from which the promoter was obtained, as a candidate Stat5-regulated gene.

19. A method for screening to identify a candidate Stat5 protein comprising:
(A) providing a nucleobase probe containing a base sequence of CTCNNNTAA or the complement thereof;
(B) contacting the probe with a polypeptide having the amino acid sequence of Stat(5) or an amino acid sequence at least 70% identical thereto, under conditions in which the polypeptide can specifically bind to the base sequence of (A), to form a complex, and removing non-specifically bound probes therefrom to leave remaining complexes, and
(C) detecting remaining complexes,
whereby detection of a remaining complex identifies the polypeptide member thereof as a candidate Stat5 protein.

20. A method for treating SMA or other SMN-deficiency in a subject, the method comprising administering to said subject a recombinant genetic vector comprising at least one copy of a host-expressible gene encoding Stat5A and comprising at least one copy of a Stathmin inhibitor.

21. The method according to claim 20, wherein the Stat5A is a constitutively activated Stat5A.

22. The method according to claim 21, wherein the constitutively activated Stat5A is a Stat5A comprising (1) Phe710 and at least one of Arg298 or Gly150, or (2) His642, according to the numbering of SEQ ID NO:3.

23. The method according to claim 21, wherein the constitutively activated Stat5A is a Stat5A comprising Phe710 and Arg298, according to the numbering of SEQ ID NO:3.

24. The method according to claim 21, wherein the Stat5 is Stat5A1*6.

25. The method according to claim 20, wherein the inhibitor in a Stathmin expression inhibitor.

26. The method according to claim 20, wherein the copy is a copy of a host-expressible Stathmin inhibitor.

27. The method according to claim 26, wherein the copy is a copy of a host-expressible Stathmin expression inhibitor.

28. The method according to claim 20, wherein the inhibitor is or encodes an RNAi nucleic acid.

29. The method according to claim 28, w the RNAi nucleic acid is a shRNA.

30. The method according to claim 28, wherein the target sequence of the antisense or RNAi nucleic acid is CGTTTGCGAGAGAAGGATA (nt728-746 of SEQ ID NO:10).

31. The method according to claim 20, wherein the vector is a viral vector

32. The method according to claim 31, wherein the viral vector is an adenoviral, adeno-associated viral, herpes viral, or lentiviral vector.
